(19) United States
(12) Patent Application Publication
(10) Pub. No.: US 2005/0256056 A1
(43) Pub. Date: Nov. 17, 2005

(54) Peptide inhibitors of HIV

(75) Inventors: Thomas W. North, Davis, CA (US); Kit Lam, Davis, CA (US); Paul A. Luciw, Davis, CA (US); Yen Duong, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California Office of Technology Transfer University of California, Oakland, CA (US)

(21) Appl. No.: 11/004,596

(22) Filed: Dec. 2, 2004

Related U.S. Application Data
(60) Provisional application No. 60/527,271, filed on Dec. 5, 2003.

(51) Int. Cl. A61K 38/08; C07K 7/06

(52) U.S. Cl. 514/15; 530/329

(57) ABSTRACT

Provided are peptides, peptide analogs, and peptide mimetics that inhibit HIV activity, pharmaceutical compositions comprising such peptides and peptide analogs, and methods of inhibiting HIV activity and/or transmission by administering the peptides and peptide analogs to a subject.

### CROSS-REFERENCES TO RELATED APPLICATIONS

This application claims benefit of Provisional Patent Application Ser. No. 60/527,271, filed Dec. 5, 2003, the content of which is incorporated herein by reference.

### STATEMENT AS TO RIGHTS TO INVENTIONS MADE UNDER FEDERALLY SPONSORED RESEARCH AND DEVELOPMENT

NOT APPLICABLE.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The present invention is directed to peptides that inhibit HIV activity, and to such HIV-inhibiting peptides identified from a peptide combinatorial library.

There is an urgent need for the development of effective anti-HIV therapeutic agents. Current highly active antiretroviral therapy (HAART) for HIV-infected individuals is targeted against the viral protease and reverse transcriptase. Although current HAART combinations can effectively reduce virus load, increase CD4 cell counts and delay disease progression, significant problems remain. These problems include: (a) drug-resistant virus variants that emerge in treated individuals, (b) antiviral drug side-effects, which can be serious and limit treatment in many cases, and (c) complexity of current treatment protocols, making it difficult to achieve full compliance by the patient. Accordingly, novel antiviral agents are needed to circumvent these serious limitations of current anti-HIV therapeutics. There is also a pressing need for effective microbicides to prevent HIV transmission.

The emergence of drug-resistant mutants of HIV has proven to be one of the greatest obstacles to successful antiviral therapy of AIDS (Deeks, S. G. J. Acquir. Immune Defic. Syndr. 26 Suppl 1:S25-S33 (2001); Loveday, C. J. Acquir. Immune Defic. Syndr. 26 Suppl 1:S10-S24 (2001); Miller, V. J. Acquir. Immune Defic. Syndr. 26 Suppl 1:S34-S50 (2001); Wainberg, M. A., and G. Friedland. JAMA 279:1977-1983 (1998)). Even HAART has been limited by the emergence of multi-drug-resistant HIV in fully compliant patients (Loveday, supra; Richman, D. D. Nature 410:995-1001 (2001); Van Vaerenbergh, K., et al. Antimicrob. Agents Chemother. 44:2109-2117 (2000). Therefore, strategies to minimize or combat resistance must be included in the design of new drugs and therapeutic approaches.

HIV Drug-Resistance

There are currently 4 classes of antiretroviral drugs that are approved for use in AIDS therapy. Three of these classes have been extensively used in highly active antiretroviral therapy (HAART): nucleoside analogs that inhibit reverse transcriptase, non-nucleoside inhibitors of reverse transcriptase and protease inhibitors (Reviewed in Richman, D. D., supra). A fourth class is represented by T20 (Fusion), a fusion/entry inhibitor that was recently approved for use in humans. Current HAART is based upon using potent combinations of these drugs, usually three or more drugs from two or more classes. Major forces leading to development of combination therapy for AIDS were the inability of individual drugs (monotherapy) to adequately reduce virus loads and the emergence of drug-resistant mutants, which was usually rapid with any single drug. Viral drug-resistance was considered the major limitation of antiretroviral drugs in the pre-HAART era (Richman, D. D. Antimicrob. Agents Chemother. 37:1207-1213 (1993); D'Aquila, R. T., et al. Ann. Intern. Med. 122:401-408 (1995); Arts, E. J., and M. A. Wainberg. Antimicrob. Agents Chemother. 40:527-540 (1996)). The development of HAART enabled suppression of virus load to undetectable levels for prolonged periods in many patients but has not eliminated problems from viral drug-resistance. The potent combinations used in HAART, when successful, decrease the rate of emergence of resistant variants due to greatly decreased viral load. Nevertheless, treatment failure is usually accompanied by emergence of HIV-1 variants that contain multiple drug-resistance mutations (Fauci, A. S. N. Engl. J Med. 341:1046-1050 (1999)). These multi-drug-resistant variants of HIV-1 can also be transmitted (Yerly, S., L., et al. Lancet 354:729-733 (1999)). The problem of drug-resistance is not limited to inhibitors of reverse transcriptase and protease. In vitro studies have also identified mutations that confer resistance to drugs targeted against the HIV-1 integrase or the entry inhibitors (Summarized in Schinazi, R. F., et al. Int. Antiviral News 7:46-69 (1999)), and resistance can occur with any new agents regardless of the viral target. Therefore, any new approach should have an integrated component aimed at minimizing viral resistance to drugs.

HIV-1 Envelope Glycoprotein Complex

Attachment of HIV-1 to a cell and fusion of the viral envelope with the cell membrane are the initial steps in infection. These processes, mediated by the envelope glycoprotein, are natural targets for development of microbicides that block infection. The HIV-1 envelope glycoprotein is synthesized as a glycoprotein precursor (gp160), which is cleaved into the surface glycoprotein (gp120) and the transmembrane domain (gp41). Gp120 and gp41 remain noncovalently attached and are present as trimers of gp120/gp41 in virions. This trimeric complex is located on the surface of HIV-1 and is anchored to the viral envelope by the C-terminal domain of gp41. Gp120 binds to cellular receptors, and the interaction of gp120 with CD4 and co-receptors (chemokine receptors such as CCR5 or CXCR4) offers potential sites for intervention with antiviral drugs. The binding of gp120 to cell surface receptors induces conformational changes in the gp120/gp41 complex which activate fusion of the viral envelope to the cell membrane (Dimitrov, D. S. Cell 91:721-730 (1997)). The ectodomain of gp41 is critical for this fusion process. Each molecule of gp41 contains two α-helical domains that are essential for fusion. The trimeric envelope glycoprotein complex contains a bundle of six helices, three that are arranged in a parallel, triple-stranded coiled-coil (the N-terminal helices); wrapped antiparallel around the outside of this are the three other (C-terminal) et-helices of the gp41 ectodomain (Chan, D. C., et al. Cell 89:263-273 (1997); Weissenhom, W., A., et al. Nature 387:426-430 (1997); Tan, K., et al. Proc. Natl. Acad. Sci. USA 94:12303-12308 (1997)). Conformational changes induced by binding of gp120 to cell surface receptors trigger formation of a transient “prehairpin intermediate” structure which resolves to a trimer of hairpins, driving membrane fusion (Chan, D. C., and P. S. Kim, Cell 93:681-684 (1998)). This mechanism and similar structures are common in fusion proteins from numerous diverse viruses (Skehel, J. J., and D. C. Wiley, Cell 95:871-874 (1998)). Specific regions of gp41 that are involved in this fusion process have been exploited in development of antiviral approaches that result in the inhibition of viral entry and inactivation of virus.

Several important features of the HIV-1 envelope proteins must be considered in approaches for development of antiviral drugs. First, the high degree of sequence variability of HIV-1 env presents a significant challenge for antiviral drug or vaccine development. Diverse HIV-1 isolates have been classified into subtypes A through K (major group, M), as well as the highly divergent groups N and O (outlier) by comparison of amino acid sequences in env or gag regions (Robertson, D. L., et al. Science 288:55-56 (2000)). Variability in env is the basis for much of the differences between subtypes of HIV-1. Envelope variability also governs HIV-1 co-receptor usage and cell tropism. The high variability of env, particularly in gp120, is also important in the evasion of antiviral immune responses (Klenerman, P., et al. Curr. Opin. Microbiol. 5:408-413 (2002); Wyatt, R., and J. Sodroski. Science 280:1884-1888 (1998)). Drugs that target more conserved regions and that are active against all or most subtypes will be much more useful than subtype-specific inhibitors of HIV-1.

There are also major differences in Env between primary isolates of HIV-1 and laboratory strains. Most notable have been the differences between primary isolates and lab strains in susceptibility to neutralizing antibodies (Burton, D. R., et al. Science 265:1024-1027 (1994)); Sullivan, N., et al. J. Virol. 69:4413-4422 (1995)). Therefore, it is important to include drug screens with several strains of HIV-1, including primary isolates, in the strategy for development of antiviral agents that interact with an envelope glycoprotein.

Entry Inhibitors

Several approaches have been taken in attempts to develop entry inhibitors of HIV. Most of these have attempted to block viral attachment to cells. Many approaches aim to specifically block interactions of gp120 with CD4 or co-receptors. Some of these are targeted to gp120, such as soluble CD4 and related derivatives. Other approaches are targeted to CD4, CR5 or CXCR4. In addition, many polyanionic inhibitors have been shown to inhibit HIV infection of cells, presumably by blocking interaction of positively charged domains of gp120 with negatively charged heparin sulfate proteoglycans on the cell surface. Although many of these attachment inhibitors have anti-HIV activity in vitro, they have not yet led to therapeutically useful drugs (reviewed in Moore, J. P., and M. Stevenson, Nat. Rev. Mol. Cell Biol. 1:40-49 (2000); Pohlmann, S., and R. W. Doms, Curr. Drug Targets Infect. Disord. 2:9-16 (2002)).

The current most promising entry inhibitors are gp41-targeted fusion inhibitors. The potential of fusion inhibitors was first demonstrated by the ability of peptides derived from gp41 to inhibit HIV-1 replication in vitro (Wild, C., et al. AIDS Res. Hum. Retroviruses 9:1051-1053 (1993)). Peptide corresponding to the C-terminal end of the gp41 ectodomain inhibit fusion/entry. One of these C-peptides, T20 (DP178), showed promise in clinical trials (Kilby, J. M., et al. Nature Med. 4:1302-1307 (1998); Kilby, J. M., et al. AIDS Res. Hum. Retroviruses 18:685-693 (2002)) and has been approved for use in AIDS therapy. T20 is a 36-amino acid peptide that includes part of the outer C-terminal a-helical domain that is involved in the fusion mechanism. T20 is able to bind to gp41 and inhibit fusion only after gp120 binds to cellular receptors, presumably exposing the prefusion intermediate for interaction with T20. A related, more potent peptide, T1249, is also in clinical trials (Tomaras, G. D., and M. L. Greenberg. Curr. Infect. Dis. Rep. 3:93-99 (2001)).

The development of other gp41-targeted peptide inhibitors has been stimulated by an understanding of the fusion mechanism and by structural details of the α-helical domains. One approach has targeted a prominent pocket located in the N-terminal coiled-coil of the prehairpin intermediate. Using a mirror-image phage display approach, Eckert et al. (Cell 99:103-115 (1999)) were able to identify cyclic D-peptides that inhibit HIV-1 entry. One of these cyclic D-peptides, D10-p5-2K, had an EC50 of 11 μM (Eckert, supra). Unfortunately, this approach does not allow direct screening of the ligands. In another approach, biased combinatorial libraries were used to identify small organic ligands that bind to this pocket (Ferrer, M., et al. Nature Struct. Biol. 6:953-960 (1999)), and these contribute to inhibition of fusion when they are attached to gp41-derived peptides. Both of these approaches validate this pocket as a potential target for anti-HIV drugs. However, current approaches are time consuming and therefore offer essentially static therapies against a rapidly changing viral target. They generally do not lend themselves to the contemporaneous development of new inhibitors of HIV variants and/mutants.

The foregoing demonstrates that there is still a need for new and better HIV inhibitors. The present invention addresses this need.

### BRIEF SUMMARY OF THE INVENTION

In one aspect, present invention provides peptides or peptide analogs and conservative variants thereof that inhibit HIV activity, the peptides or peptide analogs having the general formula: mpx¹x²ψx⁴x⁵x⁶, mpxyx¹xyψx⁴x⁵x⁶, mpx¹yψwx⁵x⁶, mpx¹x²ψwx⁵x⁶, mprx²ψx⁴x⁵x⁶, mpx²rψx⁴x⁵x⁶, mprrψx⁴x⁵x⁶, mpsyψwir, and wqnψdygy, wherein the lower case letters represent D-forms of the amino acids according to their one-letter code and ψ is a turn-promoting amino acid, including for example, L-Pro, D-Pro, L-hydroxyPro (L-Hyp), D-hydroxyPro (D-Hyp), L-Hyp(Bzl), D-Hyp(Bzl) and 0-turn mimetics, and each of x¹, x², x⁴, x⁵ and x⁶ are independently an L- or D- isomer of a naturally occurring amino acid, amino acid analog or amino acid mimetic, including L- and D- isomers of Har, Hcy, Hse, Met(O), Met (S-Me), Nle, Tau, Phg, HoPhe, Phe(2-Me), Phe(3-Me), Phe(4-Me), Phe(2-F), Phe(3-F), Phe(4-F), Phe(2-Cl), Phe(3-Cl), Phe(4-Cl), Phe(2-Br), Phe(3-Br), Phe(4-Br), Phe(2-I), Phe(3-I), Phe(4-I), Phe(2-CF₃), Phe(3-CF₃), Phe(4-CF₃), Phe(2-OMe), Phe(3-OMe), Phe(2-NO₂), Phe(3-NO₂), Phe(4-NO₂), Phe(2-CN), Phe(3-CN), Phe(4-CN), Phe(3,4-di OMe), Phe(3,4-di F), Phe(3,5-di F), Phe(2,4-di Cl), Phe(3,4-di Cl), Phe(4-N₃), Phe(4-NH₂), Phe(4-COOH), HoCit, Cit, Orn, 2-Thi, 3-Thi, Chg, Cha, Nal-2, Nal-1, Aib, Acpc, Aad, Asu, 4-Pal, 3-Pal, Pra, Abu, Nva, Dpr, Dbu, Thz, Tyr(Me), Tyr(3,5-di Br), Tyr(3,5-di I), Tyr(3,5-di NO₂), Tyr(3-NO₂), Bug, Bta, Bpa, Dpa, Deg, Dpg, Hyp, Hyp(Bzl), Acdt, Ahch, Akch, Actp, Acp, Ach, 3-Apc, 4-Apc, 4-App, Aic, Ana, Ppca, Tha, Cpa, Hle, Aoa, Aha, and Bip.

In another aspect, the invention provides methods of treating an HIV infection, methods of decreasing the frequency of transmission of an HIV infection, and methods of inhibiting HIV activity in a host, the methods comprising administering to a subject in need thereof an effective amount of one or more peptides or peptide derivatives or conservatives variants thereof that inhibit HIV activity, the peptides or peptide derivatives having the general formula: x⁻¹x¹x²ψx⁴x⁵x⁶x⁷, x⁻³x⁻²x⁻¹mpx¹x²ψx⁴x⁵x⁶, x⁻²x⁻¹mpx¹x²ψx⁴x⁵x⁶, x⁻¹mpx¹x²ψx⁴x⁵x⁶, mpx¹x²ψx⁴x⁵x⁶, mpx¹ψwx⁵x⁶, mpx¹x²ψwx⁵x⁶, mprx²ψx⁴x⁵x⁶, mpx¹rψx⁴x⁵x⁶, mprrψx⁴x⁵x⁶, mpsyψwir, and wqnψdygy, wherein the lower case letters represent D-forms of the amino acids according to their one-letter code and ψ is a turn-promoting amino acid, including for example, L-Pro, D-Pro, L-Hyp, D-Hyp, L-Hyp(Bzl), D-Hyp(Bzl) and ,B-turn mimetics; and each of x⁻³, x⁻², x⁻¹, x¹, x², x⁴, x⁵, x⁶ and x⁷ are independently an L- or D- isomer of a naturally occurring amino acid, amino acid analog or amino acid mimetic, including L- and D-isomers of Har, Hcy, Hse, Met(O), Met (S—Me), Nle, Tau, Phg, HoPhe, Phe(2-Me), Phe(3-Me), Phe(4-Me), Phe(2-F), Phe(3-F), Phe(4-F), Phe(2-Cl), Phe(3-Cl), Phe(4-Cl), Phe(2-Br), Phe(3-Br), Phe(4-Br), Phe(2-I), Phe(3-I), Phe(4-I), Phe(2-CF₃), Phe(3-CF₃), Phe(4-CF₃), Phe(2-OMe), Phe(3-OMe), Phe(2-NO₂), Phe(4-NO₂), Phe(4-NO₂), Phe(2-CN), Phe(3-CN), Phe(4-CN), Phe(3,4-di OMe), Phe(3,4-di F), Phe(3,5-di F), Phe(2,4-di Cl), Phe(3,4-di Cl), Phe(4-N₃), Phe(4-NH₂), Phe(4-COOH), HoCit, Cit, Orn, 2-Thi, 3-Thi, Chg, Cha, Nal-2, Nal-1, Aib, Acpc, Aad, Asu, 4-Pal, 3-Pal, Pra, Abu, Nva, Dpr, Dbu, Thz, Tyr(Me), Tyr(3,5-di Br), Tyr(3,5-di I), Tyr(3,5-di NO₂), Tyr(3-NO₂), Bug, Bta, Bpa, Dpa, Deg, Dpg, Hyp, Hyp(Bzl), Acdt, Ahch, Akch, Actp, Acp, Ach, 3-Apc, 4-Apc, 4-App, Aic, Ana, Ppca, Tha, Cpa, Hle, Aoa, Aha, and Bip.

In a further aspect, the invention provides peptide or peptide analogs that inhibit the activity of HIV, said peptide or peptide analog identified by a method comprising:
a) contacting in a sample an HIV target protein required for the activity of HIV with a plurality of peptide members of a combinatorial peptide library, wherein each peptide member of said library is attached to a bead, wherein each of said members of said peptide library have a formula selected from the group consisting of x⁻¹x¹x²ψx⁴x⁵x⁶x⁷, x⁻³x⁻²x⁻¹mmpx¹x²ψx⁴x⁵x⁶, x⁻²x⁻¹mpx¹x²ψx⁴x⁵x⁶, x⁻¹mpx¹x²ψx⁴x⁵x⁶, mpx¹x²ψx⁴x⁵x⁶, mpx¹yψx⁴x⁵x⁶, mpx¹yψwx⁵x⁶, mpx¹x²ψwx⁵x⁶, mprx²ψx⁴x⁵x⁶, mpx¹rψx⁴x⁵x⁶, mprrψx⁴, x⁵x⁶, mpsyψwir, and wqnψdygy, and conservative variants thereof,
wherein the lower case letters represent D-forms of the amino acids according to their one-letter code and 4 is a turn-promoting amino acid, including for example, L-Pro, D-Pro, L-Hyp, D-Hyp, L-Hyp(Bzl), D-Hyp(Bzl) and (β-turn mimetics; and each of x⁻³, x⁻², x⁻¹, x¹, x², x⁴, x⁵, x⁶ and x⁷ are independently an L- or D- isomer of a naturally occurring amino acid, amino acid analog or amino acid mimetic, including L- and D- isomers of Har, Hcy, Hse, Met(O), Met (S-Me), Nle, Tau, Phg, HoPhe, Phe(2-Me), Phe(3-Me), Phe(4-Me), Phe(2-F), Phe(3-F), Phe(4-F), Phe(2-Cl), Phe(3-Cl), Phe(4-Cl), Phe(2-Br), Phe(3-Br), Phe(4-Br), Phe(2-I), Phe(3-I), Phe(4-I), Phe(2-CF₃), Phe(3-CF₃), Phe(4-CF₃), Phe(2-OMe), Phe(3-OMe), Phe(2-NO₂), Phe(3-NO₂), Phe(4-NO₂), Phe(2-CN), Phe(3-CN), Phe(4-CN), Phe(3,4-di OMe), Phe(3,4-di F), Phe(3,5-di F), Phe(2,4-di Cl), Phe(3,4-di Cl), Phe(4-N₃), Phe(4-NH₂), Phe(4-COOH), HoCit, Cit, Orn, 2-Thi, 3-Thi, Chg, Cha, Nal-2, Nal-1, Aib, Acpc, Aad, Asu, 4-Pal, 3-Pal, Pra, Abu, Nva, Dpr, Dbu, Thz, Tyr(Me), Tyr(3,5-di Br), Tyr(3,5-di I), Tyr(3,5-di NO₂), Tyr(3-NO₂), Bug, Bta, Bpa, Dpa, Deg, Dpg, Hyp, Hyp(Bzl), Acdt, Ahch, Akch, Actp, Acp, Ach, 3-Apc, 4-Apc, 4-App, Aic, Ana, Ppca, Tha, Cpa, Hle, Aoa, Aha, and Bip;
b) isolating a bead attached to a peptide member that is bound to said HIV target protein, and
c) sequencing said peptide member.

The invention also provides methods of treating an HIV infection, methods of decreasing the frequency of transmission of an HIV infection, and methods of inhibiting HIV activity in a host, the methods comprising administering to a subject in need thereof an effective amount of one or more peptides or peptide derivatives or conservatives variants thereof, said peptides or peptide derivatives identified by the foregoing combinatorial peptide library method.

The invention further provides for pharmaceutical compositions comprising one or more of the peptides and peptide analogs.

The invention further provides for a method for prophylactically or therapeutically inhibiting an HIV infection, the method comprising administering to an individual a topical pharmaceutical composition comprising an effective amount of one or more of the peptide or peptide analogs.

In another aspect, the invention provides peptide mimetics which comprise an HIV virion-binding tridimensional structure that is functionally interchangeable to one or more of the peptide or peptide analogs.

### BRIEF DESCRIPTION OF THE DRAWINGS

FIG. 1. (a) A single positive colored bead is identified by an enzyme-linked calorimetric assay under a dissecting microscope, (b) Using a whole-cell one-bead binding assay, a positive bead is indicated by the attachment of a monolayer of human immunoblastic lymphoma cell line (OC1LY8) on the surface of the bead, and (c) Split-mix synthetic scheme for the solid phase OBOC combinatorial library method.

### DETAILED DESCRIPTION OF THE INVENTION

### DEFINITIONS

The term “contacting” includes reference to placement in direct physical association.

As used herein, “polypeptide” and “peptide” are used interchangeably and include reference to a polymer of amino acid residues. The terms apply to amino acid polymers in which one or more amino acid residues are naturally occurring amino acids and to amino acid polymers comprising one or more analogs that are structural and/or functional substitutes of naturally occurring amino acids (e.g., amino acids of non-natural chirality and chemically synthesized structural and/or functional analogs),. The terms also apply to polymers containing conservative amino acid substitutions such that the protein remains functional.

The term “residue” or “amino acid residue” or “amino acid” interchangeably refer to an amino acid that is incorporated into a protein, polypeptide, or peptide (collectively “peptide”). As used herein, amino acid refers to naturally occurring α-amino acids and their stereoisomers, as well as unnatural amino acids such as amino acid analogs, amino acid mimetics, synthetic amino acids, β-amino acids, γ-amino acids, and N-substituted glycines in either the L- or D-configuration that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Stereoisomers” of naturally occurring amino acids refers to mirror image isomers of the naturally occurring amino acids, such as L- and D-amino acid stereoisomers.

“Amino acid analogs” refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

“Amino acid mimetics” refers to chemical compounds that have a structure that is different from the general chemical structure of an amino acid, but that functions in a manner similar to a naturally occurring amino acid.

In β-amino acids, the amino acid side chain is bonded to the β-carbon atom of the carboxyl group such that there are two carbon atoms between the amino and carboxyl groups. In γ-amino acids, the amino acid side chain is bonded to the γ-carbon atom of the carboxyl group such that there are three carbon atoms between the amino and carboxyl groups. The side-chains or R-groups of β-amino acids, γ-amino acids, and N-substituted glycines can be, in either stereo configuration, the same as the side chain groups found on naturally occurring and later modified α-amino acids, as well as side chains found on amino acid analogs and amino acid mimetics.

The term “N-substituted glycine” refers to a glycine amino acid where an amino acid side chain is attached to the glycine nitrogen atom. Suitable “amino acid side chains” or “R groups” include, but are not limited to, side chains present in naturally occurring amino acids and side chains present in unnatural amino acids such as amino acid analogs, amino acid mimetics, synthetic amino acids, β-amino acids, and γ-amino acids. Examples of N-substituted glycines suitable for use in the present invention include, without limitation, N-(2-aminoethyl)glycine, N-(3-aminopropyl)glycine, N-(2-methoxyethyl)glycine, N-benzylglycine, (S)-N-(1-phenylethyl)glycine, N-cyclohexylmethylglycine, N-(2-phenylethyl)glycine, N-(3-phenylpropyl)glycine, N-(6-aminogalactosyl)glycine, N-(2-(3′-indolylethyl)glycine, N-(2-(p-methoxyphenylethyl))glycine, N-(2-(p-chlorophenylethyl)glycine, and N-[2-(p-hydroxyphenylethyl)]glycine. Such N-substituted glycines can have an L- or D-configuration. N-substituted glycine oligomers, referred to herein as “peptoids,” have been shown to be protease resistant (Miller et al., Drug Dev. Res., 35:20-32 (1995)). As such, a peptoid linker containing at least one a-amino acid having an 10 L-configuration is within the scope of the present invention.

As used herein, “peptide analog” and “peptide derivative” refer to peptides comprising one or more non-naturally occurring amino acids, unnatural amino acids, chemical amino acid analogs or amino acid mimetics of a corresponding naturally occurring amino acid and/or functional substitutes of naturally occurring amino acids.

Amino acids and analogs referred to herein are described by shorthand designations as follows in Table A. Additional unlisted amino acids and analogs will also find use in the present invention.

TABLE A
Amino Acid Nomenclature

| | | 1-letter | 1-letter |
|---|---|---|---|
| | | L-Amino | D-Amino |
| Name | 3-letter | Acid | Acid |
| Alanine | Ala | A | a |
| Arginine | Arg | R | r |
| Asparagine | Asn | N | n |
| Aspartic Acid | Asp | D | d |
| Cysteine | Cys | C | c |
| Glutamic Acid | Glu | E | e |
| Glutamine | Gln | Q | q |
| Glycine | Gly | G | g |
| Histidine | His | H | h |
| Homoarginine | Har | — | — |
| Homocysteine | Hcy | — | — |
| Homoserine | Hse | — | — |
| Isoleucine | Ile | I | i |
| Leucine | Leu | L | l |
| Lysine | Lys | K | k |
| Methionine | Met | M | m |
| Methionine sulfoxide | Met (O) | — | — |
| Methionine methylsulfonium | Met (S—Me) | — | — |
| Norleucine | Nle | — | — |
| Phenylalanine | Phe | F | f |
| Proline | Pro | P | p |
| Serine | Ser | S | s |
| Taurine | Tau | — | — |
| Threonine | Thr | T | t |
| Tryptophan | Trp | W | w |
| Tyrosine | Tyr | Y | y |
| Valine | Val | V | v |

Additional amino acid analogs used in the present invention include those shown in Table B, although those of skill in the art will readily recognize that additional unlisted amino acid analogs are applicable to the present invention. Both D- and L- isomers are included in the present invention.

TABLE B
Amino Acid Analogs

| Name | Abbreviation | Structure |
|---|---|---|
| Aminoisobutyric acid | Aib | 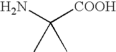 |
| 1-Amino- cyclopropanecarboxylic acid | Acpc | 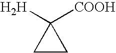 |
| 2-Aminohexanedioic acid | Aad | 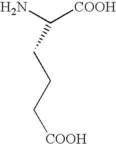 |
| 2-Aminobutyric acid | Abu | 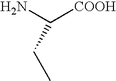 |
| 2-Aminooctanedioic acid | Asu | 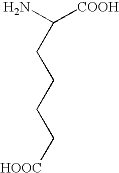 |
| Homocitrullene | HoCit | 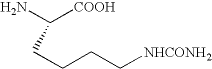 |
| Citrullene | Cit | 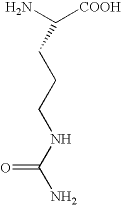 |
| Cyclohexylglycine | Chg | 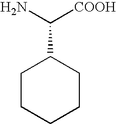 |
| Cyclohexylalanine | Cha | 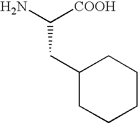 |
| Phenylglycine | Phg | 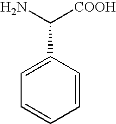 |
| 2-Methylphenylalanine | Phe(2-Me) | 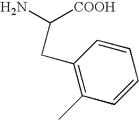 |
| 3-Methylphenylalanine | Phe(3-Me) | 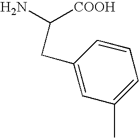 |
| 4-Methylphenylalanine | Phe(4-Me) | 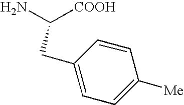 |
| 2-Fluorophenylalanine | Phe(2-F) | 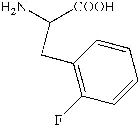 |
| 3-Fluorophenylalanine | Phe(3-F) | 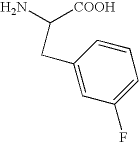 |
| 4-Fluorophenylalanine | Phe(4-F) | 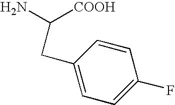 |
| 2-Chlorophenylalanine | Phe(2-Cl) | 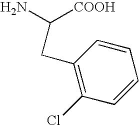 |
| 3-Chlorophenylalanine | Phe(3-Cl) | 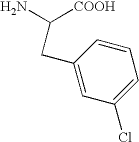 |
| 4-Chlorophenylalanine | Phe(4-Cl) | 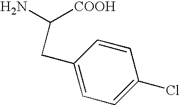 |
| 2-Bromophenylalanine | Phe(2-Br) | 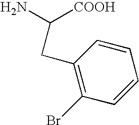 |
| 3-Bromophenylalanine | Phe(3-Br) | 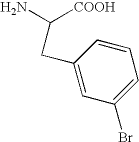 |
| 4-Bromophenylalanine | Phe(4-Br) | 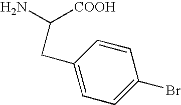 |
| 2-Iodophenylalanine | Phe(2-I) | 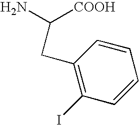 |
| 3-Iodophenylalanine | Phe(3-I) | 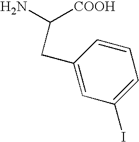 |
| 4-Iodophenylalanine | Phe(4-I) | 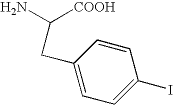 |
| 2-Trifluoromethylphenylalanine | Phe(2-CF₃) | 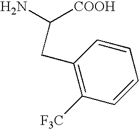 |
| 3-Trifluoromethylphenylalanine | Phe(3-CF₃) | 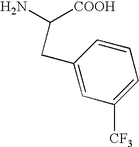 |
| 4-Trifluoromethylphenylalanine | Phe(4-CF₃) | 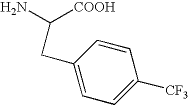 |
| 2-Methoxyphenylalanine | Phe(2-OMe) | 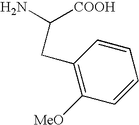 |
| 3-Methoxyphenylalanine | Phe(3-OMe) | 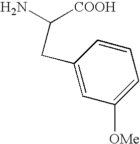 |
| 2-Cyanophenylalanine | Phe(2-CN) | 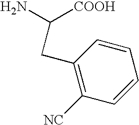 |
| 3-Cyanophenylalanine | Phe(3-CN) | 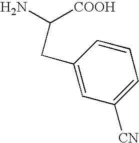 |
| 4-Cyanophenylalanine | Phe(4-CN) | 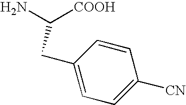 |
| 4-Aminophenylalanine | Phe(4-NH₂) | 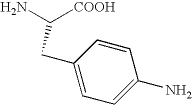 |
| 2-Nitrophenylalanine | Phe(2-NO₂) | 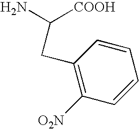 |
| 3-Nitrophenylalanine | Phe(3-NO₂) | 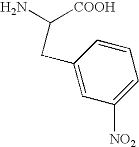 |
| 4-Nitrophenylalanine | Phe(4-NO₂) | 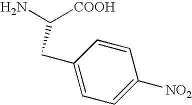 |
| 4-Azidophenylalanine | Phe(4-N₃) | 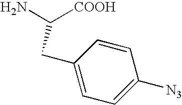 |
| 3,4-Dimethoxyphenylalanine | Phe(3,4-di OMe) | 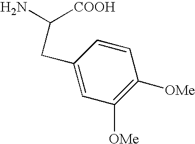 |
| 3,4-Difluorophenylalanine | Phe(3,4-di F) 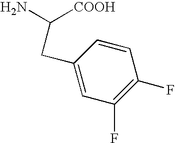 | |
| 3,5-Difluorophenylalanine | Phe(3,5-di F) 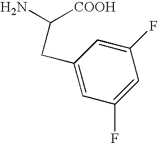 | |
| 2,4-Dichlorophenylalanine | Phe(2,4-di Cl) 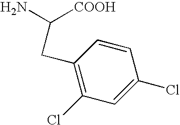 | |
| 3,4-Dichlorophenylalanine | Phe(3,4-di Cl) 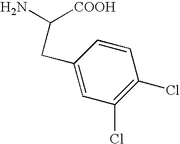 | |
| 4-Carboxyphenylalanine | Phe(4-COOH) | 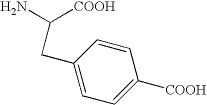 |
| Homophenylalanine | HoPhe | 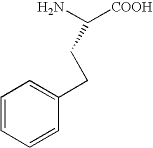 |
| 3-(2-Thienyl)alanine | 2-Thi | 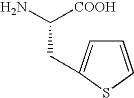 |
| 3-(3-Thienyl)alanine | 3-Thi | 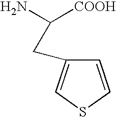 |
| 2-Naphthylalanine | Nal-2 | 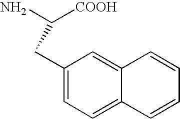 |
| 1-Naphthylalanine | Nal-1 | 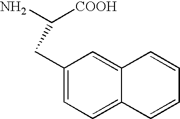 |
| 3-(4-Pyridyl)alanine | 4-Pal | 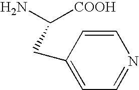 |
| 3-(3-Pyridyl)alanine | 3-Pal | 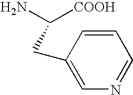 |
| Propargylglycine | Pra | 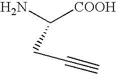 |
| Norvaline | Nva | 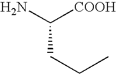 |
| 2,3-Diaminopropionic acid | Dpr | 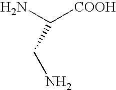 |
| 2,4-Diaminobutyric acid | Dbu | 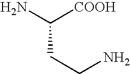 |
| Thiazolidine-4-carboxylic acid | Thz | 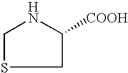 |
| Ornithine | Orn | 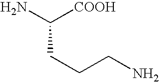 |
| 2-tert-Butylglycine | Bug | 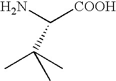 |
| O-Methyltyrosine | Tyr(Me) | 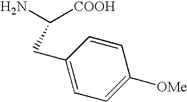 |
| 3,5-Dibromotyrosine | Tyr(di Br) | 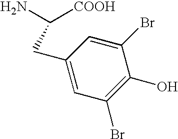 |
| 3,5-Diiodotyrosine | Tyr(3,5-di I) | 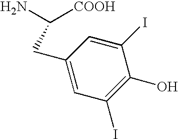 |
| 3,5-Dinitrotyrosine | Tyr(3,5-di NO₂) | 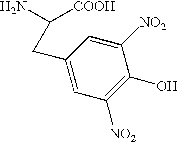 |
| 3-Nitrotyrosine | Tyr(3-NO₂) | 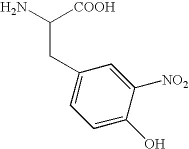 |
| Norleucine | Nle | 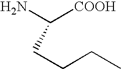 |
| 3 -Benzothienylalanine | Bta | 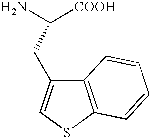 |
| 4-Benzoylphenylalanine | Bpa | 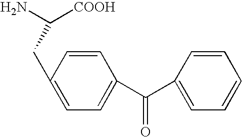 |
| 3,3-Diphenylalanine | Dpa | 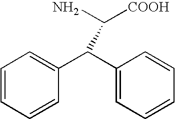 |
| Diethylglycine | Deg | 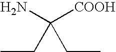 |
| Di-n-propylglycine | Dpg | 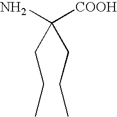 |
| Hydroxyproline | Hyp | 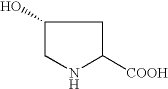 |
| O-Benzyl-hydroxyproline | Hyp(Bzl) | 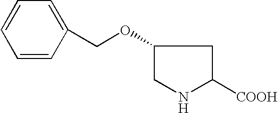 |
| 4-Amino-4-carboxy-1,1-dioxo- tetrahydrothiopyran | Acdt | 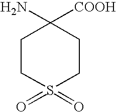 |
| 1-Amino-1-(4- hydroxycyclohexyl) carboxylic acid | Ahch | 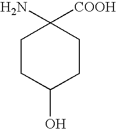 |
| 1-Amino-1-(4- ketocyclohexyl)carboxylic acid | Akch | 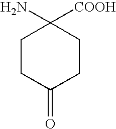 |
| 4-Amino-4- carboxytetrahydropyran | Actp | 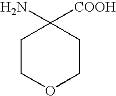 |
| 1-Amino-1-cyclopentane carboxylic acid | Acp | 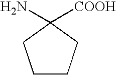 |
| 1-Amino-1-cyclohexane carboxylic acid | Ach | 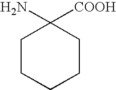 |
| 1-Amino-1-(3- piperidinyl)carboxylic acid | 3-Apc | 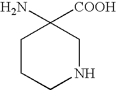 |
| 1-Amino-1-(4- piperidinyl)carboxylic acid | 4-Apc | 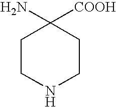 |
| 2-Amino-3-(4-piperidinyl) propionic acid | 4-App | 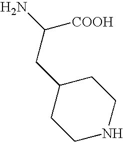 |
| 2-Aminoindane-2-carboxylic acid | Aic | 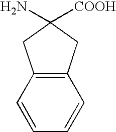 |
| 2-Amino-2-naphthylacetic acid | 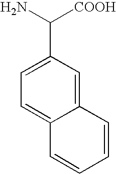 | |
| (2s, 5r)-5-Phenylpyrrolidine-2- carboxylic acid | Ppca | 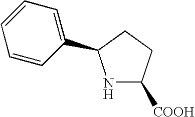 |
| 4-Thiazoylalanine | Tha | 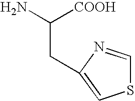 |
| β-Cyclopropylalanine | Cpa | 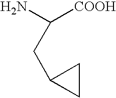 |
| Homoleucine | Hle | 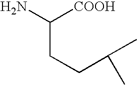 |
| 2-Aminooctanoic acid | Aoa | 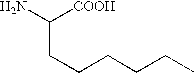 |
| 2-Aminoheptanoic acid | Aha | 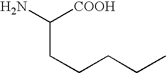 |
| 4,4′-Biphenylalanine | Bip | 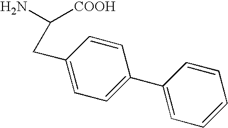 |

With respect to amino acid sequences, one of skill will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acids include, but are not limited to, naturally occurring amino acids such as α-amino acids having an L-configuration, stereorisomers of naturally occurring amino acids such as α-amino acids having a D-configuration, and unnatural amino acids such as amino acid analogs, amino acid mimetics, synthetic amino acids, β-amino acids, and γ-amino acids, in either the L- or D-configuration. For example, the unnatural amino acids of Liu and Lam (Anal. Biochem., 295:9-16 (2001)) are suitable for use in the present invention. A “conservative substitution”, refers to a change in the amino acid composition of the protein that does not substantially alter the protein's activity. Thus, “conservatively modified variations” or “conservative variants” of a particular amino acid sequence refers to amino acid substitutions of those amino acids that are not critical for protein activity or substitution of amino acids with other amino acids having functionally interchangeable or similar properties (e.g., acidic, basic, positively or negatively charged, polar or non-polar, etc.) such that the substitutions of even critical amino acids do not substantially alter activity.

Conservative substitution tables providing functionally similar amino acids are well known in the art. For example, substitutions may be made wherein an aliphatic amino acid (G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, E or D, may be substituted with its uncharged counterpart, Q or N, respectively; or vice versa. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, 1984).

A “turn-promoting amino acid,” designated as ψ herein, refers to an amino acid or analog that introduces a turn structure in a peptide, particularly along the peptide backbone. This term applies to naturally occurring and non-naturally occurring turn-promoting amino acids. Examples include L-Proline, D-Proline, L-hydroxyProline (L-Hyp), D-hydroxyProline (D-Hyp), L-Hyp(Bzl), and D-Hyp(Bzl).

A “β-turn mimetic” refers to a non-naturally occurring, chemical analog of a naturally occurring turn-promoting amino acid and functional substitutes of naturally occurring turn-promoting amino acids. β-turn mimetics of use in the present invention are described in U.S. Pat. No. 6,617,425, which is hereby incorporated herein by reference. Non-limiting examples are provided in Table D:

TABLE D
β-turn mimetics

| | | |
|---|---|---|
| | Abbre- | |
| Name | viation | Structure |
| (R,S)-3-Amino-1- carboxymethyl-2,3,4,5- tetrahydro-1H-[1]-benzazepine- 2-one | BZA | 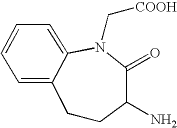 |
| 4-(2-Aminoethyl)-6- dibenzofuranpropionic acid | ADFPA | 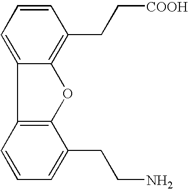 |
| (2S,6S,9S)-6-Amino-2- carboxymethyl-3-8- diazabiccyclo[4,3,0]-nonane-1,4- dione | Acdn | 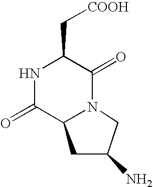 |
| 2-Oxo-3-amino-7-thia-1- azabicyclo[4.3.0]nonane-9- carboxylic acid | BTD | 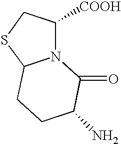 |
| (R,S)-3-amino-N-1- carboxymethyl-2-oxo-5-phenyl- 1,4-benzodiazepine | 4-BZD | 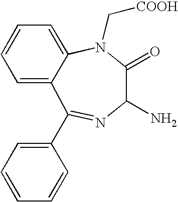 |
| 1,2,3,4-tetrahydroisoquinoline-3- carboxylic acid | Tic | 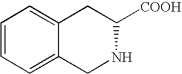 |
| (2S,5S)-5-amino-1,2,4,5,6,7- hexahydro-azepino[3,2,1- hi]indole-4-one-2-carboxylic acid | Haic | 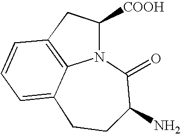 |
| [3S]-3-Amino-1- carboxymethylcaprolactame | CPL | 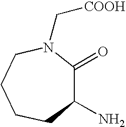 |
| (S,S)-[Pro-Leu]-spirolactamePhe | PLSP | 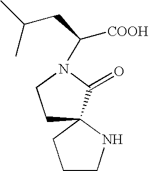 |

The term “EC₅₀” refers to the concentration of peptide that inhibits 50% viral activity, including infective activity, as measured in an in vitro assay, including a focal infectivity assay (described in Pincus, et al. Biotechniques 10:336 (1991)).

The term “HIV activity” refers to the ability of HIV to complete its infectious life-cycle, including binding, fusing, entering and replicating in a cell, release and/or lysis from a cell, as well as its transmission to another cell or another host. Inhibition of HIV activity can be accomplished by interfering with one or more steps of the HIV infectious life-cycle. HIV activity can be decreased or obliterated. Peptides and peptide analogs that decrease HIV activity, measurably decrease (e.g., by 10%, 15%, 30%, 50%) detectable indicators of HIV activity (e.g., viral titer, transmission to another cell, viral nucleic acid levels) in comparison to test samples or individuals that are not contacted with the peptides and peptide analogs.

Some peptides and peptide analogs are “virucidal” or “a virucide.” A virucidal peptide or peptide analog prevents the completion of an HIV infectious life cycle upon contact with an HIV virion, thereby obliterating its HIV activity. A virucidal peptide or peptide analog decreases the frequency of transmission and can prevent transmission of an HIV virus from a first cell to a second cell, and from a first infected host to a second host.

By “HW” is intended all HIV types, subtypes, groups and clades. HIV types include, without limitation, HIV-1 and HIV-2. HIV-1 groups include, without limitation, Groups M (main) and 0 (outgroup). Distinct HIV-1 subtypes, or “clades,” within Group M include, without limitation, clades A, B, C, D, E, F, G, H, I, J and K. Clades of HIV are described, for example, in Coffin, et al., Retroviruses, Cold Spring Harbor Laboratory Press (1997) and in Spira, et al., J. Antimicrob. Chemother. 51:229(2003).

As used herein, “administering” means oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

General

The present invention is directed to peptide inhibitors of HIV activity, and methods of inhibiting HIV activity and preventing HIV transmission by administering such peptide inhibitors, particularly peptides and peptide analogs or derivatives that are identified from combinatorial libraries.

Combinatorial Chemistry to Build Antiviral Libraries

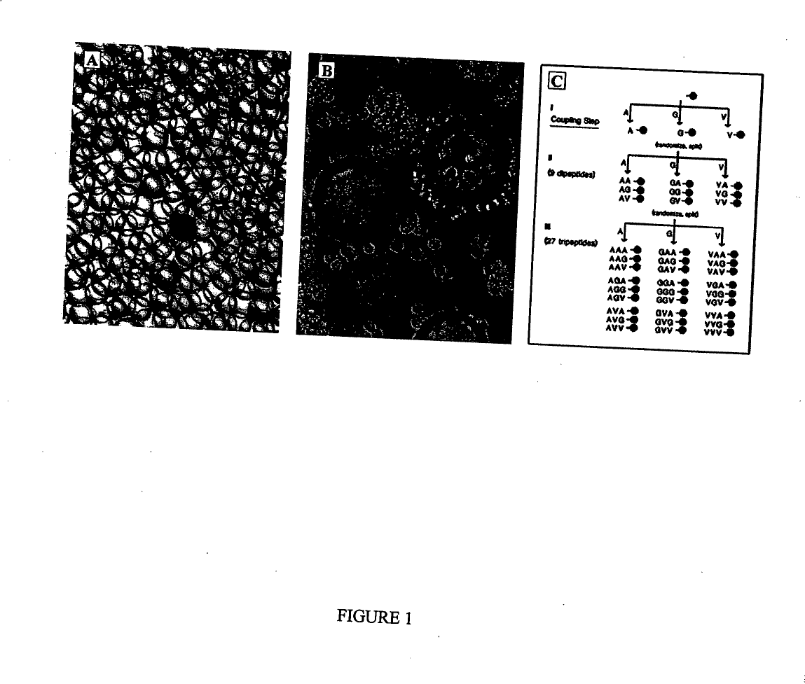

The present invention utilizes combinatorial peptide libraries, and in particular, the “one-bead-one-compound” (OBOC) combinatorial peptide library method (described in Lam, K. S., et al. Nature 354:82-84 (1991)) to identify and isolate peptide inhibitors of HIV activity. In brief, when a “split-mix” synthesis method (Lam, et al. Nature (1991), supra; Houghten, R. A., et al. Nature 354:84-86 (1991)); Furka, A., et al. Int. J. Peptide Protein Res. 37:487-493 (1991)) is used to generate a combinatorial peptide library (FIG. 1C), each bead expresses only one chemical entity (Lam, et al. Nature (1991), supra; Lam, K. S., et al. Chem. Rev. (1997), supra). Random peptide libraries of millions of beads are then screened in parallel for a specific acceptor molecule (receptor, antibody, enzyme, virus, whole cell, etc). Using an enzyme-linked colorimetric assay similar to a Western blot, the OBOC combinatorial library technique has been successfully applied to identify ligands for various targets (FIG. 1A). These include ligands for an anti-O-endorphin antibody (Lam, K. S., et al. Bioorg. Med. Chem. Lett. 3:419-424 (1993)); streptavidin (Lam, K. S., et al. Pept.: Chem., Struct. Biol., Proc. Am. Pept. Symp. 13:1005-1006 (1994)), avidin (Lam, K. S., and M. Lebl. ImmunoMethods 1:11-15 (1992)), an anti-insulin monoclonal antibody that recognizes a discontinuous epitope and MHC-Class I molecules (Lam, K. S., et al. Pept.: Chem., Struct. Biol., Proc. Am. Pept. Symp (Escom, Leiden.), indigo carmine (a small organic dye) (Lam, K. et al. Drug Development Res. 33:157-160 (1994)), and surface idiotype of B-cell lymphoma cell lines (Lam, K. S., et al. Biomed. Pept., Proteins Nucleic Acids 1:205-210 (1995)). Identified, positive beads are then physically isolated for structural determination by microsequencing using automatic Edman degradation (Lam, et al. Nature (1991), supra).

Also, screening methods can be applied to identify peptide substrate motifs from random peptide libraries. With this approach, specific and efficient peptide substrates for protein kinases have been identified (Lou, Q., et al. Cancer Res. 57:1877-1881 (1997)). Based on the peptide substrates identified from screening these libraries, several potent pseudo-substrate peptide inhibitors for p60c-src protein tyrosine kinase have been developed (Alfaro-Lopez, J., et al. J. Med Chem. 41:2252-2260 (1998); Kamath, J. R., et al. In M. Lebl and R. A. Houghten (ed.), Peptides: The wave of the future Proceedings of the Second International and the Seventeen American Peptide Symposium. American Peptide Society Publishers, San Diego. (2001) p. 551-552). Whole cell binding assays in which bead-libraries are mixed with live cells (FIG. 1B) have also been developed. Using this approach, cell surface binding peptide ligands specific for prostate and non-small cell lung cancer, and for lymphoma cell lines have been discovered (Pennington, M. E:, et al. Molecular Diversity 2:19-28 (1996); DeRoock, I. B., et al. Cancer Res. 61:3308-3313 (2001); Park, S. I., et al. Letters in Peptide Science 8:171-178 (2002); Lau, D. H., et al. Biotechnology Letters 24:497-500 (2002)).

Versatility of Synthetic Chemical Libraries

A significant advantage of the OBOC method is that it enables use of D-amino acids, unnatural amino acids, or even non-peptide moieties in the construction of libraries, which facilitate design of more proteolytically stable and therapeutically useful compounds. Accordingly, this approach can yield inhibitors with favorable stability and pharmacokinetic properties than approaches that screen for L-amino acid peptides, such as phage display libraries. Most importantly, this approach enables direct screening of these ligands rather than inferring desired molecules as has been done with the mirror image phage display library method (Eckert, D. M., et al. Cell 99:103-115 (1999)). Another advantage of the OBOC combinatorial library method is that each compound is spatially separated on individual beads, and multiple different motifs or binding sites can be identified (Wu, J., et al. J. Comb. Chem. Highthroughput Screening, 5:83-91 (2002)). This gives the combinatorial approach great versatility.

For peptide-bead libraries, structure determination of the positive bead is straightforward. The peptide-bead can be directly microsequenced with an automatic sequencer using Edman chemistry. In contrast, peptidomimetic-bead libraries with non-sequenceable building blocks require a different strategy for structure determination. A novel encoding system developed for structural determination of peptidomimetic libraries has been developed (see, for example, “A Novel Encoding Method for “One-Bead One Compound” Combinatorial Libraries”, filed on Mar. 28, 2003 as Attorney Docket No. 02307W-131500US, which is hereby incorporated herein by reference).

Combinatorial libraries offer a unique opportunity to identify new lead compounds and/or to identify new drug targets for AIDS therapy. Importantly, they provide a rational approach for design of drugs that can be “evolved” in response to emergence of drug-resistant variants of HIV. These approaches allow rapid screening of millions of molecules to enable identification of molecules that bind to specific targets. Also, these approaches can be used to develop specific inhibitors. The methods of the present invention can also be used to modify inhibitors to obtain derivatives that are active against drug-resistant HIV variants and mutants.

Entry Inhibitors

In the present invention, the OBOC combinatorial library method is applied to identify novel inhibitors that target HIV virion components. Targets, including the glycoproteins gp120 and gp41, are critical for attachment and for fusion-entry of virus into cells. The identification of novel inhibitors that exert their activities at the pre-entry step are of particular interest. Inhibiting attachment and/or fusion-entry is also a logical point for development of inhibitors to block infection for potential use as prophylactic microbicides. Several sites on gp120 and gp41 may present favorable targets for intervention with antiviral drugs. The approaches of the present invention are advantageous in that identified inhibitors are not limited to a single site, and inhibitors of drug-resistant HIV variants can be efficiently identified.

Innovation & Significance

Combinatorial libraries offer a unique opportunity to identify new lead compounds and/or to identify new drug targets for blocking HIV infection and AIDS therapy. These methods yield inhibitors of HIV that are comparable or greater in potency than several of the approved anti-HIV drugs (e.g., nucleoside analogs, reviewed in Richman, D. D. Nature 410:995 (2001)). The optimization procedures facilitate the efficient development of lead compounds into therapeutically useful drugs. Because the peptide and peptide analog HIV activity inhibitors of the present invention block viral attachment onto and/or entry into cells, they are well suited for development of microbicides to prevent HIV-1 transmission. The approach can also be applied to any other virus.

One of the most significant features of the combinatorial approach is that it enables an innovative strategy to combat viral drug-resistance. Drug-resistance has been and remains the greatest impediment to long-term success with AIDS therapy. The versatility of the combinatorial chemistry approach of the present invention enables efficient re-selection of compounds from defined libraries by screening with ogp140 containing drug-resistance mutations (Srivastava, I. K., et al., J. Virol. 76:2835-2847 (2002)). In this manner, one can “re-evolve” a peptide or peptide analog inhibitor in response to emergence of a resistant variant. A “re-evolved” inhibitor can inhibit wild-type virus along with a mutant or drug-resistant virus. Also, a mixture of two or more inhibitors can be effective against both wild-type and mutant viruses. A combinatorial approach will facilitate targeting wild-type HIV-1, major drug-resistant variants and other HIV-1 mutants.

This approach to combat resistance should limit any drug-resistance to variants that emerge less frequently. These mutants are more likely to have reduced fitness. This strategy enables development of a drug or drug combination that will select for mutants that are attenuated. Such a regimen could be used long-term because in this case the drug-resistance mutations would be beneficial.

### Description of the Embodiments

Peptides and Peptide Analogs

In one aspect, the present invention provides peptides and peptide analogs and conservative variants thereof that inhibit or obliterate HIV activity, the peptides and peptide analogs having the general formula: mpx¹x²ψx⁴x⁵x⁶, mpx¹yψx⁴x⁵x⁶, mpx¹yψwx⁵x⁶, mpx¹x²ψwx⁵x⁶, mprx²ψx⁴x⁵x⁶, mpx¹rψx⁴x⁵x⁶, and mprrψx⁴x⁵x⁶, wherein the lower case letters represent D-forms of the amino acids according to their one-letter code and ψ is a turn-promoting amino acid, including for example, L-Pro, D-Pro, L-Hyp, D-Hyp, L-Hyp(Bzl), D-Hyp(Bzl) and β-turn mimetics, and each of x¹, x², x⁴, x⁵ and x⁶ are independently an L- or D- isomer of a naturally occurring amino acid, amino acid analog or amino acid mimetic, including L- and D- isomers of Har, Hcy, Hse, Met(O), Met (S-Me), Nle, Tau, Phg, HoPhe, Phe(2-Me), Phe(3-Me), Phe(4-Me), Phe(2-F), Phe(3-F), Phe(4-F), Phe(2-Cl), Phe(3-Cl), Phe(4-Cl), Phe(2-Br), Phe(3-Br), Phe(4-Br), Phe(2-I), Phe(3-I), Phe(4-I), Phe(2-CF₃), Phe(3-CF₃), Phe(4-CF₃), Phe(2-OMe), Phe(3-OMe), Phe(2-NO₂), Phe(3-NO₂), Phe(4-NO₂), Phe(2-CN), Phe(3-CN), Phe(4-CN), Phe(3,4-di OMe), Phe(3,4-di F), Phe(3,5-di F), Phe(2,4-di Cl), Phe(3,4-di Cl), Phe(4-N₃), Phe(4-NH₂), Phe(4-COOH), HoCit, Cit, Orn, 2-Thi, 3-Thi, Chg, Cha, Nal-2, Nal-1, Aib, Acpc, Aad, Asu, 4-Pal, 3-Pal, Pra, Abu, Nva, Dpr, Dbu, Thz, Tyr(Me), Tyr(3,5-di Br), Tyr(3,5-di I), Tyr(3,5-di NO₂), Tyr(3-NO₂), Bug, Bta, Bpa, Dpa, Deg, Dpg, Hyp, Hyp(Bzl), Acdt, Ahch, Akch, Actp, Acp, Ach, 3-Apc, 4-Apc, 4-App, Aic, Ana, Ppca, Tha, Cpa, Hle, Aoa, Aha, and Bip.

Usually the peptides and peptide analogs are 6-mers, 7-mers, 8-mers, 9-mers, 10-mers, 11-mers or 12-mers, although they may comprise more or less amino acid residues, as appropriate. Typically, the peptides or peptide analogs are 6 to 8 residues in length. More, typically, the peptides or peptide analogs are 8-mers. Generally, the peptide and peptide analogs will contain a turn-promoting amino acid residue in a middle residue position, having from 2-8, more typically from 3-6 or 3-5, and most typically from 3-4 amino acid residues on either side of the turn-promoting amino acid.

The turn-promoting amino acid can be a naturally occurring amino acid or a non-naturally occurring amino acid. In one embodiment, the turn-promoting amino acid is selected from L-Pro, D-Pro, L-Hyp, D-Hyp, L-Hyp(Bzl) or D-Hyp(Bzl). In one embodiment, the turn-promoting amino acid is a β-turn mimetic. In a further embodiment, the β-turn mimetic is selected from the group consisting of BZA, ADFPA, Acdn, BTD, 4-BZD, Tic, Haic, CPL and PLSP.

In certain preferred embodiments, the peptide and peptide analog sequences have a formula selected from the group consisting of mpayψwir, mpsavψwir, mpsyvψair, mpsyψwar and mpsyψwia. Usually, the peptides and peptide analogs will have a motif comprising critical residues in anchoring positions. For example, in one embodiment, the peptide and peptide analog sequences have a general formula selected from the group consisting of mprx²ψx⁴x⁵x⁶, mpx¹rψx⁴x⁵x⁶, and mprrψx⁴x⁵x⁶. In other preferred embodiments, the peptide and peptide analog sequences have a general formula selected from the group consisting of mpx¹yψx⁴x⁵x⁶, mpx¹yψwx⁵x⁶, mpx¹x²ψwx⁵x⁶. In a preferred embodiment, the peptide or peptide analog has the formula mpsyψwir. In another aspect, the peptide or peptide analog has the formula wqnψdygy.

The peptide and peptide analogs can be comprised of naturally occurring amino acid residues, non-naturally occurring amino acid residues, functional or structural chemical amino acid analogs, amino acid mimetics, and combinations thereof. As appropriate, the peptides or peptide analogs can also have post-synthesis modifications. In certain embodiments, the peptides are methylated, acetylated, alkylated, arylated, halogenated, or otherwise appropriately substituted.

The peptides or peptide analogs can be linear or cyclic. In certain embodiments, the peptides or peptide analogs are dimerized, trimerized, otherwise multimerized, or cyclized as multimers or monomers. Usually, the peptides and peptide analogs are linear. In certain embodiments, multimerization of peptides and peptide analogs is accomplished by direct intermolecular bond formation, for instance through amide or disulfide bonds. Likewise, cyclization of one or more peptides or peptide analogs can be through direct inter and/or intramolecular bond formation.

In certain embodiments, the multimerization or cyclization of one or more peptides or peptide analogs is accomplished through a linker, generally via covalent bond formation. As used herein, a linker refers to any molecule containing a chain of atoms, e.g., carbon, nitrogen, oxygen, sulfur, etc., that serves to link a first peptide monomer with one or more additional peptide monomers. Examples of linkers include aminobutyric acid, aminocaproic acid, 7-aminoheptanoic acid, 8-aminocaprylic acid, lysine, iminodiacetic acid, polyoxyethylene, glutamic acid, etc. In further embodiments, linkers can additionally comprise one or more β-alanines or other amino acids as spacers. Suitable linkers can be cleavable or non-cleavable. Exemplified peptide coupling reagents include isobutyl chloroformate (IBCF), oxalyl chloride or disuccinimidyl carbonate (DSC). Additional suitable chemical linkers are commercially available from Pierce Biotechnology, Rockford, Ill. (www.piercenet.com).

The peptides and peptide analogs of the present invention inhibit or obliterate HIV activity in an infected host. Further, the peptides and peptide analogs can prophylactically and therapeutically inhibit or prevent transmission of an HIV virus from a first infected individual to a second individual. Peptides and peptide analogs of particular interest inhibit or prevent HIV attachment to a host cell and/or inhibit or prevent HIV entry into a host cell. Usually, the peptides and peptide analogs bind to an HIV envelope glycoprotein, including gp120, and/or an HIV transmembrane subunit glycoprotein, including gp41, and inhibit or prevent HIV attachment and/or entry into a host cell. Peptides and peptide analogs that bind to rapidly mutating HIV proteins are of particular interest. In certain embodiments, the peptides and peptide analogs bind to a binding region of an HIV envelope glycoprotein that is not the CD4 binding region, or a co-receptor binding region or a region known to fuse with a cell membrane.

In a preferred embodiment, the peptides and peptide analogs are virucidal. Virucidal peptides and peptide analogs decrease the frequency of and can prevent the transmission of an HIV virus from a first infected individual to a second individual. Within a host, a virucidal peptide or peptide analog decreases the frequency of and can prevent the transmission of an HIV virus from a first infected cell to a second cell.

Of particular interest are multimerized monomers of peptides or peptide analogs, wherein each peptide monomer inhibits or prevents a distinct function of HIV activity or binds to a distinct binding region on the same or different HIV proteins. A binding region can be a continuous sequence segment or based on the tridimensional structure of one or more closely proximate non-continuous sequence segments. For instance, in one embodiment the peptide or peptide analog is a dimer comprised of a first monomer that inhibits or prevents HIV attachment and a second monomer that inhibits or prevents HIV entry into a cell. In another preferred embodiment, the peptide or peptide analog is a dimer comprised of a first monomer that binds to gp120 and a second monomer that binds to gp41.

The peptides and peptide analogs bind to an envelope protein of one or more types or subtypes of HIV virions, e.g., to an envelope protein of one or more HIV clades. Preferred peptides and peptide analogs bind to an envelope protein of at least two, three, four, five or more HIV clades. In certain embodiments the peptides and peptide analogs inhibit or prevent the activity of an HIV variant or mutant, usually a drug-resistant HIV variant or mutant. Of particular interest are those peptides and peptide analogs that inhibit the activity of major, or most commonly identified, drug resistant HIV mutants. Typically, the peptides and peptide analogs inhibit HIV activity in vitro with an EC₅₀ of less than about 50 μM or 40 μM, more preferably less than about 30 μM or 20 μM, and most preferably less than about 15 μM, 10 μM or 5 μM or less.

Methods of Making the Peptides and Peptide Analogs

Split-Mix Synthesis Methodology

In one preferred embodiment, the peptides and peptide analogs of the present invention can be prepared using combinatorial peptide libraries, and in particular, the “one-bead-one-compound” (OBOC) combinatorial peptide library method (described in Lam, K. S., et al. Nature 354:82-84 (1991)), typically prepared with a “split-mix” synthesis method (Lam, et al. Nature (1991), supra; Houghten, R. A., et al. Nature 354:84-86 (1991)); Furka, A., et al. Int. J. Peptide Protein Res. 37:487-493 (1991)). Preferred methods for preparing peptides and peptide analogs of the present invention are further described in, for example, “A Novel Encoding Method for “One-Bead One Compound” Combinatorial Libraries”, filed on Mar. 28, 2003 as Attorney Docket No. 02307W-131500US and Preparation and “Application of Encoded Bead Aggregates in Combinatorial Chemistry”, filed on Mar. 28, 2003 as Attorney Docket No. 02307W-132600US, and in U.S. Pat. Nos. 6,090,912; 5,858,670; 5,840,485; and 5,650,489, each of which is hereby incorporated herein by reference.

Generally, the synthesis of peptide and peptide analog libraries of synthetic test peptides and peptide analogs via a split-mix methodology comprises repeating the following steps, schematically depicted in FIG. 1(C): (i) dividing the selected solid support (e.g., beads) into a number of portions which is at least equal to the number of different amino acids to be linked; (ii) chemically linking one and only one of the amino acids of the synthetic test peptide or peptide analog with one and only one of the portions of the solid support from step (i), preferably making certain that the chemical link-forming reaction is driven to completion to the fullest extent possible; (iii) thoroughly mixing the solid support portions containing the growing synthetic test peptide or peptide analog; (iv) repeating steps (i) through (iii) a number of times equal to the number of amino acids in each of the synthetic test peptide or peptide analog of the desired library, thus growing the synthetic test peptide or peptide analog; (v) removing any protecting groups that were used during the assembly of the synthetic test peptide or peptide analog on the solid support. The repetition of steps (i)-(iii) (see step (iv)) will naturally result in growing the synthetic test peptide or peptide analog.

In one embodiment, enough support particles are used so that there is a high probability that every possible structure of the synthetic test peptide or peptide analog is present in the library. Such a library is referred to as a “complete” library. To ensure a high probability of representation of every structure requires use of a number of supports in excess, e.g., by five-fold, twenty-fold, etc., according to statistics, such as Poisson statistics, of the number of possible species of peptides and peptide analogs. In another embodiment, especially where the number of possible structures exceeds the number of supports, not every possible structure is represented in the library. Such “incomplete” libraries are also very useful.

Screeninig

The peptide or peptide analogs of the present invention can be identified by a method comprising:
a) contacting in a sample an HIV target protein required for the activity of HIV with a plurality of peptide members of a combinatorial peptide library, wherein each peptide member of said library is attached to a solid support, for example, a bead,
b) isolating a bead attached to a peptide member that is bound to said HIV target protein, and
c) sequencing said peptide member.

Usually, the HI target protein is an HIV envelope protein, including a gp120 protein or a gp41 protein or a combination thereof. Preferably, peptides and peptide analogs of interest are identified by contacting them with a soluble HIV envelope protein, for example, ogp140, which contains gp120 and the ectodomain of the gp41 subunit in a trimeric form with a configuration that closely approximates native HIV envelope glycoprotein in membranes of virions and infected cells (Srivastava, I. K., et al. J. Virol. 76:2835-2847 (2002); and Earl, P. L., et al. J. Virol. 75:645-653 (2001)).

The HIV target protein is attached to a detection moiety, for instance a radioactive isotope (e.g., ³²P, ¹²⁵I), a fluorescent reporting group, an enzyme (e.g., alkaline phosphatase), a member of a binding pair (e.g., biotin-avidin, biotin-streptavidin), or a magnetic bead (including those commercially available from Dynal Biotech of Lake Success, N.Y. (www.dynalbiotech.com) and from Miltenyi Biotec of Auburn, Calif. (www.miltenyibiotec.com)). In certain embodiments, the HIV target protein is attached to a fluorescent reporting group, including fluoroscein (green), Texas Red (red), DAPI (blue), or BOPIDI. Additional suitable fluorescent reporting groups are commercially available from Molecular Probes of Eugene, Oreg. (www.probes.com) and from Epoch Biosciences of Bothell, Wash. (www.epochbio.com). In preferred embodiments, the HIV target protein is attached to a member of a binding pair, for instance, biotin.

As appropriate, the peptide and peptide analogs may be incubated with additional moieties to produce detectable signal. For instance, when using an HIV target protein attached to a first binding pair member, the peptides and peptide analogs are contacted with the second binding pair member, wherein the second binding pair member is bound to a reporter group, such as a fluorophore, an enzyme or a magnetic bead. If using an enzyme, such as alkaline phosphatase, the peptides and peptide analogs are subsequently contacted with an appropriate colorimetric enzyme substrate, such as 5-bromo-4-chloro-3-indolylphosphate (BCIP), NBT, New Fuchsin, p-NPP or Fast Red, to produce a detectable signal. Suitable alkaline phosphatase substrates are commercially available from, for example, Sigma Aldrich Chemicals (St Louis, Mo.), and Vector Laboratories (Burlingame, Calif.). Chemiluminscent alkaline phosphatase substrates can also be used. The peptides and peptide analogs can then be immobilized in a medium, for instance in low-melting agarose, and those bound to the HIV target protein can be physically isolated from peptides and peptide analogs that are not bound to the HIV target protein. Peptides or peptide analogs bound to an HIV target protein labeled with a fluorescent reporting group are identified using a fluorescence microscope with appropriate excitation filters. Peptides or peptide analogs bound to an HIV target protein labeled with a binding pair member-enzyme reporting group complex and contacted with a colorimetric substrate can be identified using a transmissive scanner. Positive beads can be physically isolated using techniques known in the art.

Isolated peptides are washed with a denaturant, for instance, 6M guanidine-HCl, to remove bound proteins. The sequences of isolated peptides are then identified by conventional sequencing techniques, including Edman degradation, using an automated protein sequencer.

Peptides and peptide analogs that bind to an HIV target protein of interest are then screened for their effectiveness in inhibiting HIV activity in vitro. One assay for evaluating HIV inhibiting activity in vitro is a focal infectivity assay, described by Pincus, et al. Biotechniques 10:336 (1991). Preferred peptides and peptide analogs will inhibit HIV activity in vitro at a concentration of about 50 μM or less, more preferably at about 40 μM, 30 μM, 25 μM or 20 μM or less, and more preferably at about 15 μM, 10 μM or 5 μM or less.

In a further aspect, the invention provides peptide or peptide analogs that inhibit the activity of HIV, said peptide or peptide analog identified by a method comprising:
a) contacting in a sample an HIV target protein required for the activity of HIV with a plurality of peptide members of a combinatorial peptide library, wherein each peptide member of said library is attached to a bead, wherein each of said members of said peptide library have a formula selected from the group consisting of x⁻¹x¹x²ψx⁴x⁵x⁶x⁷, x⁻³x⁻²x⁻¹mpx¹x²ψx⁴x⁵x⁶, x⁻²x⁻¹mpx¹x²ψx⁴x⁵x⁶, x⁻¹mpx¹x²ψx⁴x⁵x⁶, mpx¹x²ψx⁴x⁵x⁶, mpx¹yψx⁴x⁵x⁶, mpx¹yψwx⁵x⁶, mpx¹x²ψwx⁵x⁶, mprx²ψx⁴x⁵x⁶, mpx¹rψx⁴x⁵x⁶, mprrψx⁴x⁵x⁶, mpsyψwir, and wqnψdygy, and conservative variants thereof,
wherein the lower case letters represent D-forms of the amino acids according to their one-letter code and 4, is a turn-promoting amino acid, including for example, L-Pro, D-Pro, L-Hyp, D-Hyp, L-Hyp(Bzl), D-Hyp(Bzl) and fl-turn mimetics; and each of x⁻³, x⁻², x⁻¹, x¹, x², x⁴, x⁵, x⁶ and x⁷ are independently an L- or D- isomer of a naturally occurring amino acid, amino acid analog or amino acid mimetic, including L- and D- isomers of Har, Hcy, Hse, Met(O), Met (S-Me), Nle, Tau, Phg, HoPhe, Phe(2-Me), Phe(3-Me), Phe(4-Me), Phe(2-F), Phe(3-F), Phe(4-F), Phe(2-Cl), Phe(3-Cl), Phe(4-Cl), Phe(2-Br), Phe(3-Br), Phe(4-Br), Phe(2-I), Phe(3-I), Phe(4-I), Phe(2-CF₃), Phe(3-CF₃), Phe(4-CF₃), Phe(2-OMe), Phe(3-OMe), Phe(2-NO₂), Phe(3-NO₂), Phe(4-NO₂), Phe(2-CN), Phe(3-CN), Phe(4-CN), Phe(3,4-di OMe), Phe(3,4-di F), Phe(3,5-di F), Phe(2,4-di Cl), Phe(3,4-di Cl), Phe(4-N₃), Phe(4-NH₂), Phe(4-COOH), HoCit, Cit, Orn, 2-Thi, 3-Thi, Chg, Cha, Nal-2, Nal-1, Aib, Acpc, Aad, Asu, 4-Pal, 3-Pal, Pra, Abu, Nva, Dpr, Dbu, Thz, Tyr(Me), Tyr(3,5-di Br), Tyr(3,5-di I), Tyr(3,5-di NO₂), Tyr(3-NO₂), Bug, Bta, Bpa, Dpa, Deg, Dpg, Hyp, Hyp(Bzl), Acdt, Ahch, Akch, Actp, Acp, Ach, 3-Apc, 4-Apc, 4-App, Aic, Ana, Ppca, Tha, Cpa, Hle, Aoa, Aha, and Bip;
b) isolating a bead attached to a peptide member that is bound to said HIV target protein, and
c) sequencing said peptide member.

The invention also provides methods of treating an HIV infection, methods of decreasing the frequency of transmission of an HIV virus from a first infected individual to a second individual, and methods of inhibiting HIV activity in a host, the methods comprising administering to a subject in need thereof an effective amount of one or more peptides or peptide derivatives or conservatives variants thereof, said peptides or peptide derivatives identified by the by the foregoing combinatorial peptide library method.

Use in Designing a Peptide Mimetic

In a further aspect, the present invention provides the use of the peptides and peptide analogs in the design of a pharmaceutical compound which is modelled to resemble the three dimensional structure, the steric size, and/or the charge distribution of a peptide or peptide analog of interest, wherein the compound has the functionally interchangeable property of binding to an HIV virion in the same manner as the peptide or peptide analog of interest. By “peptide mimetic” or “peptidomimetic” is intended a molecule that mimics the biological activity of a peptide but is no longer peptidic in chemical nature or that is partially peptidic in nature, such as pseudo-peptides, semi-peptides and peptoids. Whether completely or partially non-peptide, peptidomimetics provide a spatial arrangement of reactive chemical moieties that closely resembles the three-dimensional arrangement of active groups in the peptide on which the peptidomimetic is based. By studying the functional interactions between the peptide or peptide analogs and the HIV virion or HIV protein to which they bind, compounds which contain functional groups arranged in such a manner that they reproduce those interactions can be designed.

There are several steps commonly taken in the design of a peptidomimetic using a peptide of interest as a lead compound. First, the particular parts of the compound that are critical and/or important in inhibiting HIV activity are determined. In the case of a peptide, this can be done by systematically varying the amino acid residues in the peptide, e.g., by substituting each residue in turn, for instance, with alanine. The parts or residues constituting the active region of the compound are known as its “pharmacophore.” Once the pharmacophore has been found, its structure is modelled according to its physical properties, e.g. stereochemistry, bonding, size and/or charge, using data from a range of sources, e.g., spectroscopic techniques, X-ray diffraction data and NMR. Computational analysis, similarity mapping (which models the charge and/or volume of a pharmacophore, rather than the bonding between atoms) and other techniques can be used in this modelling process. Alternatively, the three-dimensional structure of the peptide or peptide analog bound to an HIV virion or HIV protein is modelled, particularly where binding induces conformational changes.

A template molecule is then selected onto which chemical groups which mimic the pharmacophore are grafted. The template molecule and the chemical groups grafted onto it are selected so that the mimetic is easy to synthesize, is likely to be pharmacologically acceptable, and does not degrade in vivo, while retaining the biological activity of the lead compound. The mimetic or mimetics found by this approach are then screened for HIV inhibitory activity, for instance in an in vitro focal infectivity assay. Further optimization or modification is then carried out to arrive at one or more final mimetics for further testing or optimization, e.g., in vitro, in vivo or clinical testing. Mimetics of the peptides and peptide analogs and their use in therapy form a further aspect of the invention.

Methods of Treating an Individual

In another aspect, the invention provides methods of treating an HIV infection, methods of decreasing the frequency of transmission of an HIV infection, and methods of inhibiting HIV activity in a host, the methods comprising administering to a subject in need thereof an effective amount of one or more peptides or peptide derivatives or conservatives variants thereof that inhibit HIV activity, the peptides or peptide derivatives having the general formula: x⁻¹x¹x²ψx⁴x⁵x⁶x⁷, x⁻³x⁻²x⁻¹mpx¹x²ψx⁴x⁵x⁶, x⁻²x⁻¹mpx¹x²ψx⁴x⁵x⁶, x⁻¹mpx¹x²ψx⁴x⁵x⁶, mpx¹x²ψx⁴x⁵x⁶, mpx¹yψx⁴x⁵x⁶, mpx¹yψwx⁵x⁶, mpx¹x²ψwx⁵x⁶, mprx²ψx⁴x⁵x⁶, mpx¹rψx⁴x⁵x⁶, mprrψx⁴x⁵x⁶, mpsyψwir, and wqnψdygy, wherein the lower case letters represent D-forms of the amino acids according to their one-letter code and ψ is a turn-promoting amino acid, including for example, L-Pro, D-Pro, L-Hyp, D-Hyp, L-Hyp(Bzl), D-Hyp(Bzl) and -turn mimetics; and each of x⁻³, x⁻², x⁻¹, x¹, X², x⁵, x⁶ and x⁷ are independently an L- or D- isomer of a naturally occurring amino acid, amino acid analog or amino acid mimetic, including L- and D- isomers of Har, Hcy, Hse, Met(O), Met (S-Me), Nle, Tau, Phg, HoPhe, Phe(2-Me), Phe(3-Me), Phe(4-Me), Phe(2-F), Phe(3-F), Phe(4-F), Phe(2-Cl), Phe(3-Cl), Phe(4-Cl), Phe(2-Br), Phe(3-Br), Phe(4-Br), Phe(2-I), Phe(3-I), Phe(4-I), Phe(2-CF₃), Phe(3-CF₃), Phe(4-CF₃), Phe(2-OMe), Phe(3-OMe), Phe(2-NO₂), Phe(3-NO₂), Phe(4-NO₂), Phe(2-CN), Phe(3-CN), Phe(4-CN), Phe(3,4-di OMe), Phe(3,4-di F), Phe(3,5-di F), Phe(2,4-di Cl), Phe(3,4-di Cl), Phe(4-N₃), Phe(4-NH₂), Phe(4-COOH), HoCit, Cit, Orn, 2-Thi, 3-Thi, Chg, Cha, Nal-2, Nal-1, Aib, Acpc, Aad, Asu, 4-Pal, 3-Pal, Pra, Abu, Nva, Dpr, Dbu, Thz, Tyr(Me), Tyr(3,5-di Br), Tyr(3,5-di I), Tyr(3,5-di NO₂), Tyr(3-NO₂), Bug, Bta, Bpa, Dpa, Deg, Dpg, Hyp, Hyp(Bzl), Acdt, Ahch, Akch, Actp, Acp, Ach, 3-Apc, 4-Apc, 4-App, Aic, Ana, Ppca, Tha, Cpa, Hle, Aoa, Aha, and Bip.

In a further aspect, the invention provides methods of preventing an HIV infection, methods of preventing transmission of an HIV infection, and methods of obliterating HIV activity in a host, the methods comprising administering to a subject in need thereof an effective amount of one or more virucidal peptides or peptide derivatives or conservatives variants thereof, the peptides or peptide derivatives having the general formula: x⁻¹x¹x²ψx⁴x⁵x⁶x⁷, x⁻³x⁻²x⁻¹mpx¹x²ψx⁴x⁵x⁶, x⁻²x⁻¹mpx¹x²ψx⁴x⁵x⁶, x⁻¹mpx¹x²ψx⁴x⁵x⁶, mpx¹x²ψx⁴x⁵x⁶, mpx¹yψx⁴x⁵x⁶, mpx¹yψwx⁵x⁶, mpx¹x²ψwx⁵x⁶mprx²ψx⁴x⁵x⁶, mpx¹rψx⁴x⁵x⁶, mprrψx⁴x⁵x⁶, mpsyψwir, and wqnψdygy, wherein the lower case letters represent D-forms of the amino acids according to their one-letter code and ψ is a turn-promoting amino acid, including for example, L-Pro, D-Pro, L-Hyp, D-Hyp, L-Hyp(Bzl), D-Hyp(Bzl) and β-turn mimetics; and each of x⁻³, x⁻², x⁻¹, x¹, x², x⁴, x⁵, x⁶ and x⁷ are independently an L- or D-isomer of a naturally occurring amino acid, amino acid analog or amino acid mimetic, including L- and D- isomers of Har, Hcy, Hse, Met(O), Met (S-Me), Nle, Tau, Phg, HoPhe, Phe(2-Me), Phe(3-Me), Phe(4-Me), Phe(2-F), Phe(3-F), Phe(4-F), Phe(2-Cl), Phe(3-Cl), Phe(4-Cl), Phe(2-Br), Phe(3-Br), Phe(4-Br), Phe(2-I), Phe(3-I), Phe(4-1), Phe(2-CF₃), Phe(3-CF₃), Phe(4-CF₃), Phe(2-OMe), Phe(3-OMe), Phe(2-NO₂), Phe(3-NO₂), Phe(4-NO₂), Phe(2-CN), Phe(3-CN), Phe(4-CN), Phe(3,4-di OMe), Phe(3,4-di F), Phe(3,5-di F), Phe(2,4-di Cl), Phe(3,4-di Cl), Phe(4-N₃), Phe(4-NH₂), Phe(4-COOH), HoCit, Cit, Om, 2-Thi, 3-Thi, Chg, Cha, Nal-2, Nal-1, Aib, Acpc, Aad, Asu, 4-Pal, 3-Pal, Pra, Abu, Nva, Dpr, Dbu, Thz, Tyr(Me), Tyr(3,5-di Br), Tyr(3,5-di I), Tyr(3,5-di NO₂), Tyr(3-NO₂), Bug, Bta, Bpa, Dpa, Deg, Dpg, Hyp, Hyp(Bzl), Acdt, Ahch, Akch, Actp, Acp, Ach, 3-Apc, 4-Apc, 4-App, Aic, Ana, Ppca, Tha, Cpa, Hle, Aoa, Aha, and Bip.

The peptides and peptide analogs of the present invention are particularly suited for inhibiting or preventing HIV activity and for decreasing the frequency of or preventing HIV transmission of one or more HIV types and/or subtypes (clades), HIV mutants and HIV variants, and especially those that are unresponsive to currently administered anti-HIV therapies, for instance, currently used HAART therapies. Preferred peptides and peptide analogs can inhibit or prevent the HIV activity or transmission of at least two, three, four, five, six or more HIV subtypes or clades. Preferred peptides and peptide analogs are HIV virucides.

In some embodiments, the peptides and peptide analogs are administered therapeutically to an HIV infected individual. In some embodiments, the peptides and peptide analogs are administered prophylactically to an uninfected individual.

The peptides and peptide analogs are administered to a subject through any route of administration that allows contact with an HIV virion, and particularly with an HIV envelope protein. Usually the peptides and peptide analogs are formulated for oral administration, but can also be administered parenterally, as appropriate. For instance, the peptides can be administered by injection (intraveneously, intramuscularly, subcutaneously, intrathecally), or given transdermally, intraocularly, as an inhalant (pulmonary delivery) or intranasally. Usually, the peptides are administered orally, intravenously or topically.

In one preferred embodiment, the peptides and peptide analogs are administered topically. Accordingly, the invention further provides for a method for prophylactically or therapeutically decreasing the frequency of or preventing the transmission of an HIV infection, the method comprising topically administering to an individual a pharmaceutical composition comprising an effective amount of one or more of the peptide or peptide analogs of the present invention. Peptides and peptide analogs formulated in topical pharmaceutical compositions can be prophylactically or therapeutically applied to an individual's skin or mucous membranes to decrease the frequency of or prevent the transmission of HIV infection. The topical composition is preferably introduced into the vagina, at about the time of, and preferably prior to, sexual intercourse, but may also be administered to other topically accessible skin or mucous membrane. Topical compositions can also be administered to the penis, the rectum or the mouth of an individual. The manner of administration is preferably designed to obtain direct contact of the compositions of the invention with an HIV virion. Preferably, the peptides and peptide analogs administered for topical delivery are virucidal.

An efficacious or effective amount of one or more peptides or peptide derivatives is determined by applying methods known to those in the art, generally by first administering a low dose or small amount of peptide, and then incrementally increasing the administered dose until a desired effect of inhibited HIV activity is observed in the treated subject, with minimal or no toxic side effects. Applicable methods for determining an appropriate dose and dosing schedule for administration of one or more of the peptides and peptide analogs of the present invention are described, for example, in Goodman and Gilman 's The Pharmacological Basis of Therapeutics, 10^{th} Ed., Hardman, Limbird and Goodman-Gilman, Eds., McGraw-Hill (2001), and in Remington: The Science and Practice of Pharmacy, 21^{st} Ed., Gennaro, Ed., Lippencott Williams & Wilkins (2003). Further guidance is provided in Pharmaceutical Dosage Forms and Drug Delivery Systems, 7^{th} Ed., by Ansel, Allen and Popovich, Lippencott Williams & Wilkins (2000).

A desired effect of inhibited HIV activity in a host can be measured in any of a number of ways known to those in the art. Typically, changes in HIV activity in a host are observed by measuring numbers of CD4⁺ T cells (CD4⁺ counts), HIV RNA plasma levels, usually from infected cells, such as CD4⁺ T cells, before and after treatment with a peptide or peptide analog. Usually several HIV activity measurements are taken at designated time periods subsequent to commencing administration of the peptides or peptide analogs, for instance, bi-weekly, weekly, bimonthly, monthly, every 2^{nd} or 3^{rd} month, semi-annually, annually, as is appropriate. Human clinical trials of treatments against HIV activity described by Lawrence, et al. N. Engl. J. Med. 349:827 (2003); Squires, et al., Ann. Intern. Med. 139:313 (2003); Abrams, et al., Ann. Intern. Med. 139:258 (2003); Havlir, et al., J. Virol. 77:11212 (2003) are instructive. Preferred peptides and peptide analogs decrease HIV activity in a host, for instance, by the measured indicators of increasing CD4⁺ counts or decreasing HIV RNA levels, by at least 5-10%, more preferably by at least 15%, 20%, 25% or 30%, and most preferably by at least 35%, 40%, 45%, 50% or more.

In certain embodiments the peptides and peptide analogs are administered to enhance the efficacy of chemotherapeutics currently administered to HIV infected individuals. For instance, the peptides and peptide analogs can be administered in combination with one or more HIV reverse transcriptase inhibitors and/or HIV protease inhibitors.

Pharmaceutical Compositions

The invention also provides for pharmaceutical compositions comprising the peptides of the present invention. Generally, the pharmaceutical compositions of the present invention comprise one or more peptides or peptide analogs that inhibit HIV activity, as described herein, or pharmaceutically acceptable salts thereof, together with one or more pharmaceutically acceptable carriers, diluents and/or excipients. The pharmaceutical compositions are prepared according to methods known in the art based on the desired route of administration (e.g., oral, intravenous, intramuscular, subcutaneous, intravaginal, intrarectal, intranasal). For instance, depending on the intended route of administration, the pharmaceutical compositions can be formulated as, for example, a liquid, gel, semi-solid, solid, cream or ointment. The compositions can be aqueous, oil-based, emulsified or dry (e.g., a compressed powder). In certain embodiments, the peptide pharmaceutical compositions are prepared in a controlled and/or extended-release formulation (see, for example, U.S. Pat. Nos. 6,235,712; 6,187,330; 6,180,608; 6,159,490 and 6,068,850, each of which is hereby incorporated herein by reference). In certain embodiments, the peptides and peptide analogs are encapsulated for delivery. Preferred pharmaceutical compositions allow for delivery of an efficacious amount of the peptides and peptide analogs to HIV virion repository sites in a host, and contact of the peptide and peptide analogs with an HIV virion. General principles applicable for designing pharmaceutical compositions comprising peptides are found, for example, in Goodman and Gilman's The Pharmacological Basis of Therapeutics, supra; Remington: The Science and Practice of Pharmacy, supra; Shahrokh, et al. Eds., Therapeutic Protein and Peptide Formulation and Delivery, American Chemical Society (1997) and in Hashida, et al., Trends and Future Perspectives in Peptide and Protein Drug Delivery (Drug Targeting and Delivery), Taylor & Francis (1995). Further guidance is provided in Pharmaceutical Dosage Forms and Drug Delivery Systems, supra. Exemplified pharmaceutical compositions of applicable for delivery of the peptide and peptide analogs of the present invention are described in U.S. Pat. Nos. 6,565,879; 6,541,606; 6,506,730; 6,387,406; 6,346,242; and U.S. patent Publication Nos. 2003/0171296 and 2003/0017203, each of which is hereby incorporated herein by reference.

In one embodiment, the peptides and peptide analogs are prepared in pharmaceutical compositions formulated for oral administration. Formulations suitable for oral administration can consist of liquid solutions, such as an effective amount of one or more of the peptides or peptide analogs dissolved in diluents, such as water, saline, or fruit juice; capsules, sachets or tablets, each containing a predetermined amount of the active ingredient, as solid, granules or freeze-dried cells; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Suitable formulations for oral delivery can also be incorporated into synthetic and natural polymeric microspheres, or other means to protect the agents of the present invention from degradation within the gastrointestinal tract (see, for example, Wallace et al., Science 260, 912-915, 1993).

In another embodiment, the peptides and peptide analogs are prepared in pharmaceutical compositions formulated for intravenous delivery. Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

In one embodiment, the peptides and peptide analogs are prepared in pharmaceutical compositions formulated for topical administration, for instance, in a cream, a paste, a gel, a foam, an ointment, a spray, a lubricant, an emulsion or suspension. In a preferred embodiment, the pharmaceutical compositions formulated for topical administration comprise one or more peptides and peptide analogs. In a preferred embodiment, the pharmaceutical compositions formulated for topical administration comprise one or more virucidal peptides and peptide analogs that decrease the frequency or prevent the transmission of an HIV virus from a first infected individual to a second individual. Topical microbicidal preparations suitable for formulating pharmaceutical compositions comprising the peptides and peptide analogs of the present invention are described in Turpin, Expert Opin. Investig. Drugs 11: 1077 (2002); Garg, et al., AIDS Patient Care Stds 17:17 (2003); Ketas, et al., AIDS Res Hum Retroviruses 19:177(2003); in U.S. Pat. Nos. 6,267,985; 6,248,363 and 5,747,058, and in U.S. patent Publication No. 2003/0049320, each of which is hereby incorporated herein by reference. Additional topical microbicidal formulations that can find use in preparing pharmaceutical compositions for the topical delivery of the peptides and peptide analogs are described in U.S. Pat. Nos. 6,635,242; 6,596,763; 6,566,095; 6,500,460; 6,428,790; 6,420,336; 6,376,504; 6,350,784; 6,165,493, each of which is hereby incorporated herein by reference.

In topical formulations, usually the peptides or peptide analogs are included in about 0.1, 0.2, 0.5, 1.0 or 2.0 wt %, but can be included in as much as 5, 10, 15 or 20 wt % of the total formulation, or more. The peptides and peptide analogs are formulated with one or more pharmaceutically acceptable carriers. For topical applications, the pharmaceutically acceptable carrier may additionally comprise organic solvents, emulsifiers, gelling agents, moisturizers, stabilizers, other surfactants, wetting agents, preservatives, time release agents, and minor amounts of humectants, sequestering agents, dyes, perfumes, and other components commonly employed in pharmaceutical compositions for topical administration. Solid dosage forms for topical administration include suppositories, powders, and granules. In solid dosage forms, the compositions may be admixed with at least one inert diluent such as sucrose, lactose, or starch, and may additionally comprise lubricating agents, buffering agents and other components well known to those skilled in the art.

Peptide formulations suitable for vaginal administration can be presented as pessaries, tampons, creams, gels, pastes, foams, or spray formulas. Similarly, the active ingredient can be combined with a lubricant as a coating on a condom. Indeed, preferably, the active ingredient is applied to any contraceptive device, including, but not limited to, a condom, a diaphragm, a cervical cap, a vaginal ring and a sponge. Formulations for rectal administration can be presented as a suppository with a suitable base comprising, for example, cocoa butter or a salicylate. Other articles and delivery systems of this type will be readily apparent to those skilled in the art.

### EXAMPLES

The following examples are offered to illustrate, but not to limit, the claimed invention.

### Example 1

### Screening to Identify Peptides That Bind to ogp140

This example demonstrates the success of using a one-bead-one-compound combinatorial peptide library to identify peptides that bind to HIV envelope proteins and inhibit HIV activity. Preliminary screening studies were performed with biotinylated-ogp140 on a library of L-amino acid cyclic peptides to optimize conditions before the screening using more expensive D-amino acid and unnatural amino acid peptide libraries. Ogp140 derived from the HIV-1 SF162 strain and gp120 subunit, for use as a control in differentiating anti-envelope inhibitors that bind to either gp120 or gp41, was generously provided by Dr. Indresh Srivastava of Chiron Corporation. The peptide library had the following general chemical structure: cXXXXXXc, wherein c=D-cysteine, X=all L-amino acids except Cys. The optimal concentrations of glycoproteins for our screening assays were determined to be 0.25-1.25 μg/ml for ogp140 and 1-5 μg/ml for gp120. In order to visualize beads with bound ogp140 or gp120, these proteins were biotinylated so that an enzyme-linked colorimetric assay could be used to identify positive beads. Biotinylation was performed with Sulpho-NHS-biotin at a 5:1 molar ratio of biotin to protein. Reactions were carried out for both 3 minutes and 10 minutes to obtain different levels of biotinylation and were stopped with 2 M Tris-HCl, pH 7.4. The proteins were dialyzed for 4-5 hr to remove excess biotin. From this library, identified 9 cyclic peptides were identified that bind to ogp140.

Ogp140 binding was then screened with a D-amino acid library and a similar library consisting of D-amino acids except for one L-proline. These libraries were: xxxxpxxxx and xxxPxxx where x=all D-amino acids except cysteine, p=D-proline, P=L-proline (Uppercase letters refer to L-amino acids and lowercase to D-amino acids). Both of these libraries have a proline in the middle of the peptide chain. This results in a somewhat constrained, turn structure in most of the peptides. The approximate number of beads that were screened with each library was 120,000. Both libraries were screened with ogp140 at concentrations of 0.25 and 1.25 μg/ml. Beads were incubated for 2 hr with ogp140 and then were treated with streptavadin-alkaline phosphatase. Beads were then immobilized in 1% low melting agarose, stained with BCIP (5-bromo-4-chloro-3-indolylphosphate), and positive beads were visualized with a transmissive scanner. To minimize the number of false positive beads, an image subtraction system was used (see, U.S. patent Publication No. 2003/0139322, hereby incorporated herein by reference). Twenty two positive beads were detected and they were isolated using a dissecting microscope. Isolated beads were washed with 6M guanidine-HCl, pH 1.0 to remove bound proteins, then peptides on individual beads were sequenced with Edman chemistry using an automated protein sequencer.

After re-synthesis of 16 of these as soluble peptides, 13 were tested for ability to inhibit the NL4-3 clone of HIV-1 (the other 3 were insoluble). For these studies a focal infectivity assay was carried out using HeLa H1-JC.37 cells. H1-JC.37 cells are HeLa cells that naturally express the CXCR4 receptor and that have been engineered to express CD4 and CCR5 receptors (Platt, et al. J. Virol. 72:2855 (1998)). Dose-response curves were performed in triplicate experiments with 6 determinations per drug concentration in each experiment. Mean EC₅₀ values are presented in Table 1. None of these peptides inhibited growth of HeLa H1-JC.37 cells at the concentrations tested (up to 100 μM). The purity of the most potent peptide, # 16, was analyzed by HPLC, and this revealed a major peak and two smaller peaks (which presumably were deletion peptides). The purified peak fraction was re-tested for anti-HIV activity, and the EC₅₀ was determined to be 8 μM. The identity of the biologically active peptide has been confirmed with mass spectrometry.

TABLE 1
Peptides that bind of ogp140 and EC₅₀ values
for inhibition of HIV-1 NL4-3.

| | peptide | |
|---|---|---|
| peptide # | sequence | EC50 (μM) |
| 1 | ffdipiqvm | ND* |
| 2 | pahwpnwqd | 45 |
| 3 | yyqhplkgv | >100 |
| 4 | ptrfpmlge | >100 |
| 5 | fefPmvlf | ND* |
| 6 | whmPtfls | >100 |
| 7 | ywhPmeal | >100 |
| 8 | infPhyqs | >100 |
| 9 | wnpPfqqd | >100 |
| 10 | dwnPhlfi | >100 |
| 11 | twqPfhsi | >100 |
| 12 | gfyPefyh | >100 |
| 13 | hmnPfyyq | >100 |
| 14 | yyqPfdhv | 55 |
| 15 | irtyPgqi | ND* |
| 16 | mpsyPwir | 17 |

*ND = Not Determined due to lack of solubility

This success with the first libraries that were screened demonstrates the value of this approach.

### Example 2

### Optimization by Alanine Walk

This example demonstrates the optimization of a lead peptide identified by screening a combinatorial peptide library. An “alanine walk” experiment was conducted with our lead peptide mpsyψwir (#16, Table 1). In this experiment peptides were synthesized with replacement of each position, one at a time, with D-Ala. In addition the L-Pro was replaced with D-Pro (#10) or with L-Ala (#5). These peptides were analyzed for antiviral activity against HIV-1 NL4-3 strain. Results are shown in Table 2.

TABLE 2
Alanine Walk with mpsyPwir (peptide #16)

| Peptide # | Peptide | EC50 (μM) |
|---|---|---|
| 1 | apsyPwir | >100 |
| 2 | masyPwir | >100 |
| 3 | mpayPwir | 14 |
| 4 | mpsaPwir | 80 |
| 5 | mpsyAwir | >100 |
| 6 | mpsyawir | >100 |
| 7 | mpsyPair | 51 |
| 8 | mpsyPwar | 11 |
| 9 | mpsyPwia | 32 |
| 10 | mpsypwir | 15 |
| 16 | mpsyPwir | 8 |

These results demonstrate that D-Met in position 1, D-Pro in position 2 and L-Pro in position 5 are crucial for antiviral activity. Replacement of any of these residues with D-Ala led to a complete loss of activity. D-Tyr at position 4 and D-Trp at position 6 are also very important as replacement of these residues led to a 6-10 fold decrease in antiviral activity. Interestingly, the L-Pro at position 5 can be replaced by a D-Pro and still retain considerable activity, but replacing it by D-Ala or L-Ala led to a complete lost of activity, suggesting that a turn structure is crucial. The data in Table 2 were obtained against HIV-1 NL4-3 strain, which uses the CXCR4 co-receptor. Importantly, both mpsyψwir and mpsyψwar (peptides #16 and #8 in Table 3) also inhibit SF 162 strain of HIV-1 (EC50 values <10 μM), which uses the CCR5 co-receptor. Thus, our lead compound is active against both an X4 and an R5 strain of HIV-1. The peptides of Table 2 were re-synthesized on beads. Beads that have the active peptides (#3, 8, 10 and 16) stain more intensively in binding assays with ogp140, suggesting that antiviral activity of this series of peptides correlates with affinity for binding to ogp140.

### Example 3

### Optimization of Lead Compound: mpsyψwir

This example demonstrates the identification of a common motif in peptides selected from a secondary library that was screened for binding to ogp140 under higher stringency. Based on these results of the alanine walk, the following libraries for binding to ogp140 were synthesized and screened:

| | |
|---|---|
| mpxxψxxx | (8-mer) |
| xmpxxψxxx | (9-mer) |
| xxmpxxψxxx | (10-mer) |
| xxxmpxxψxxx | (11-mer) |

where x=33 different D- and unnatural amino acids and ψ=amino acids that promote turns (e.g. L-Pro, D-pro, L-Hyp, D-Hyp, L-Hyp(Bzl), D-Hyp(Bzl) and β-turn mimetics). The bolded amino acids, D-methionine and D-proline, are fixed.

These secondary libraries were screened with ogp140 under higher stringency (lower protein concentrations of both ogp140 and streptavidin-alkaline phosphatase (ST-AP)). The concentrations of ogp140 used in this screen were 0.06 mg/mL and 0.3 mg/mL. These concentrations are 4-fold lower than those used in the original screen (0.25 mg/mL and 1.25 mg/mL). The amount of ST-AP was lowered from 1:15,000 in the original screen to 1:25,000 in this screen. Approximately 75,000 beads from each library were screened.

From the 8-mer library, there were 34 positive beads, 6 from the 9-mer library, 8 from the 10-mer library, and 3 from the 11-mer library. As these numbers indicate, the 8-mer library produced the greatest number of peptides able to bind to ogp140. This library also yielded beads that stained with the greatest intensity, a preliminary indication of strong binding. From the 34 positive beads of the 8-mer library, the 12 darkest beads were selected for sequence analyses and the results are in Table 3. Based upon staining intensity of these beads, these peptides appear to bind to ogp140 with higher affinity than peptide, mpsyψwir. An interesting motif, mprr at the N-termini, was revealed from the sequence data. Two of these 12 peptides had D-arginine at positions 3 and 4 and an additional 8 of these peptides had D-arg at either position 3 or 4.

TABLE 3
Sequence Analysis of Peptides from Screening of Secondary Libraries

| | 1 | 2 | 3 | 4 | 5 | 6 | 7 | 8 |
|---|---|---|---|---|---|---|---|---|
| pep # | (N₈) | (N₇) | (N₆) | (N₅) | (N₄) | (N₃) | (N₂) | (N₁) |
| 1 | m | p | w | r | L-pro | h | Phg | h |
| 2 | m | p | r | r | BZA | a | w | v |
| 3 | m | p | r | Phe(4-Me) | Hyp | y | h | h |
| 4 | m | p | r | r | Acdn | f | d | HoCit |
| 5 | m | p | HoCit | w | BTD | r | l | a |
| 6 | m | p | Phe(4-Me) | r | BTD | Phe(4-Me) | HoCit | h |
| 7 | m | p | Thi | r | 4-BZD | y | h | s |
| 8 | m | p | Chg | r | Tic | h | p | HoCit |
| 9 | m | p | r | h | Tic | h | Phe(4-Me) | q |
| 10 | m | p | r | m | Hyp | Chg | HoCit | Cha |
| 11 | m | p | D-Nal-2 | g | Haic | m | Cha | k |
| 12 | m | p | Aib | r | 4-BZD | i | y | t |

The pepides of Table 3, and the tetrapetide mprr, are then re-synthesized in solution form and evaluated for anti-HIV activity.

### Example 4

### Identification of a Peptide Inhibitor that Binds to ogp140 but not to ogp120

A subtraction screen with ogp120 followed by a screen for binding to ogp140 produced 19 positive beads from 2 different libraries. Twelve of the peptides were re-synthesized in solution form, purified and tested for anti-HIV activity. The results are listed in Table 4:

TABLE 4
EC₅₀ Values for Peptides Found to Bind to ogp140 but not to ogp120

| peptide # | sequence | EC₅₀ (μM) |
|---|---|---|
| 1 | yhgPifih | *ND |
| 2 | fydPyplf | *ND |
| 3 | wdfPyghg | >100 |
| 4 | infPhfeh | *ND |
| 5 | wshPyylm | >100 |
| 6 | wshPyylt | >100 |
| 7 | nwhPafmd | >100 |
| 8 | wqnPdygy | 5 |
| 9 | inhPvfef | >100 |
| 10 | fygPeffs | >100 |
| 11 | ehfPfvhv | >100 |
| 12 | nmyPsyly | >100 |

*ND = no data due to peptide insolubility

Peptide #8, wqnψdygy, inhibited HIV with an EC₅₀ value of 5 μM, which is slightly more potent than our first lead compound, peptide mpsyψwir. Alanine-walk and secondary libraries are then used to optimize this peptide using the approach described above.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A peptide or peptide analog having a formula selected from the group. consisting of:
mpx¹x²ψx⁴x⁵x⁶, mpx¹yψx⁴x⁵x⁶, mpx¹yψwx⁵x⁶, mpx¹x²ψwx⁵x⁶, mprx²ψx⁴x⁵x⁶, mpx¹rψx⁴x⁵x⁶, and mprrψx⁴x⁵x⁶
and conservative variants thereof, wherein the lower case letters represent D-forms of the amino acids according to their one-letter code and 4 is a turn-promoting amino acid selected from the group consisting of L-Pro, D-Pro, L-Hyp, D-Hyp, L-Hyp(Bzl), D-Hyp(Bzl) and β-turn mimetics, and each of x¹, x², x⁴, x⁵ and x⁶ are independently selected from L-amino acids, D-amino acids, and L- and D- isomers of Har, Hcy, Hse, Met(O), Met (S-Me), Nle, Tau, Phg, HoPhe, Phe(2-Me), Phe(3-Me), Phe(4-Me), Phe(2-F), Phe(3-F), Phe(4-F), Phe(2-Cl), Phe(3-Cl), Phe(4-Cl), Phe(2-Br), Phe(3-Br), Phe(4-Br), Phe(2-I), Phe(3-I), Phe(4-I), Phe(2-CF₃), Phe(3-CF₃), Phe(4-CF₃), Phe(2-OMe), Phe(3-OMe), Phe(2-NO₂), Phe(3-NO₂), Phe(4-NO₂), Phe(2-CN), Phe(3-CN), Phe(4-CN), Phe(3,4-di OMe), Phe(3,4-di F), Phe(3,5-di F), Phe(2,4-di Cl), Phe(3,4-di Cl), Phe(4-N₃), Phe(4-NH₂), Phe(4-COOH), HoCit, Cit, Orn, 2-Thi, 3-Thi, Chg, Cha, Nal-2, Nal-1, Aib, Acpc, Aad, Asu, 4-Pal, 3-Pal, Pra, Abu, Nva, Dpr, Dbu, Thz, Tyr(Me), Tyr(3,5-di Br), Tyr(3,5-di I), Tyr(3,5-di NO₂), Tyr(3-NO₂), Bug, Bta, Bpa, Dpa, Deg, Dpg, Hyp, Hyp(Bzl), Acdt, Ahch, Akch, Actp, Acp, Ach, 3-Apc, 4-Apc, 4-App, Aic, Ana, Ppca, Tha, Cpa, Hle, Aoa, Aha, and Bip, wherein said peptide inhibits HIV activity.

2. The peptide or peptide analog of claim 1, wherein ψ is selected from the group consisting of L-Pro, D-Pro, L-Hyp, D-Hyp, L-Hyp(Bzl), D-Hyp(Bzl), BZA, ADFPA, Acdn, BTD, 4-BZD, Tic, Haic, CPL and PLSP.

3. The peptide or peptide analog of claim 1, said peptide or peptide analog having a formula selected from the group consisting of mpayψwir, mpsaψwir, mpsyψair, mpsyψwar and mpsyψwia.

4. The peptide or peptide analog of claim 1, wherein said peptide inhibits HIV activity of one or more HIV clades.

5. The peptide or peptide analog of claim 1, wherein said peptide interrupts the completion of an HIV life cycle.

6. The peptide or peptide analog of claim 5, wherein said peptide inhibits HIV attachment to a cell.

7. The peptide or peptide analog of claim 5, wherein said peptide inhibits HIV entry into a cell.

8. The peptide or peptide analog of claim 5 wherein said peptide is a virucide.

9. The peptide or peptide analog of claim 1, wherein said peptide binds to an HIV envelope glycoprotein gp120.

10. The peptide or peptide analog of claim 1, wherein said peptide binds to an HIV transmembrane subunit gp41.

11. The peptide or peptide analog of claim 1, wherein said peptide binds to a site on an HIV envelope protein that does not bind to a CD4 molecule, a co-receptor molecule, or fuse with a host cell.

12. The peptide or peptide analog of claim 1, wherein said peptide inhibits HIV activity in vitro with an EC₅₀ of less than 20 μM.

13. The peptide or peptide analog of claim 1, wherein said peptide is isolated from a combinatorial peptide library.

14. A peptide or peptide analog having a formula selected from the group consisting of:
mpsyψwir and wqnψdygy
and conservative variants thereof, wherein the lower case letters represent D-forms of the amino acids according to their one-letter code and ψ is a turn-promoting amino acid selected from the group consisting of L-Pro, D-Pro, L-Hyp, D-Hyp, L-Hyp(Bzl), D-Hyp(Bzl) and β-turn mimetics, wherein said peptide inhibits HIV activity.

15. The peptide or peptide analog of claim 14, having the formula mpsyψwir and conservative variants thereof, wherein ψ is selected from the group consisting of L-Pro, D-Pro, L-Hyp, D-Hyp, L-Hyp(Bzl), D-Hyp(Bzl), BZA, ADFPA, Acdn, BTD, 4-BZD, Tic, Haic, CPL and PLSP.

16. The peptide or peptide analog of claim 14, having the formula wqnψdygy and conservative variants thereof, wherein ψ is selected from the group consisting of L-Pro, D-Pro, L-Hyp, D-Hyp, L-Hyp(Bzl), D-Hyp(Bzl), BZA, AtIFPA, Acdn, BTD, 4-BZD, Tic, Haic, CPL and PLSP.

17. A method of treating HIV infection, said method comprising administering to a subject in need thereof, an effective amount of one or more peptides or peptide derivatives having a formula selected from the group consisting of
x⁻³x⁻²x⁻¹mpx¹x²ψx⁴x⁵x⁶, x⁻²x⁻¹mpx¹x²ψx⁴x⁵x⁶, mpx¹x²ψx⁴x⁵x⁶, x⁻¹x¹x²ψx⁴x⁵x⁶x⁷, and conservative variants thereof,
wherein
the lower case letters represent D-forms of the amino acids according to their one-letter code and ψ is a turn-promoting amino acid selected from the group consisting of L-Pro, D-Pro, L-Hyp, D-Hyp, L-Hyp(Bzl), D-Hyp(Bzl) and β-turn mimetics; and each of x⁻³, x⁻², x⁻¹, x¹, x², x⁴, x⁵, x⁶ and x⁷ are independently selected from L-amino acids, D-amino acids, and L- and D- isomers of Har, Hcy, Hse, Met(O), Met (S-Me), Nle, Tau, Phg, HoPhe, Phe(2-Me), Phe(3-Me), Phe(4-Me), Phe(2-F), Phe(3-F), Phe(4-F), Phe(2-Cl), Phe(3-Cl), Phe(4-Cl), Phe(2-Br), Phe(3-Br), Phe(4-Br), Phe(2-I), Phe(3-I), Phe(4-I), Phe(2-CF₃), Phe(3-CF₃), Phe(4-CF₃), Phe(2-OMe), Phe(3-OMe), Phe(2-NO₂), Phe(3-NO₂), Phe(4-NO₂), Phe(2-CN), Phe(3-CN), Phe(4-CN), Phe(3,4-di OMe), Phe(3,4-di F), Phe(3,5-di F), Phe(2,4-di Cl), Phe(3,4-di Cl), Phe(4-N₃), Phe(4-NH₂), Phe(4-COOH), HoCit, Cit, Orn, 2-Thi, 3-Thi, Chg, Cha, Nal-2, Nal-1, Aib, Acpc, Aad, Asu, 4-Pal, 3-Pal, Pra, Abu, Nva, Dpr, Dbu, Thz, Tyr(Me), Tyr(3,5-di Br), Tyr(3,5-di I), Tyr(3,5-di NO₂), Tyr(3-NO₂), Bug, Bta, Bpa, Dpa, Deg, Dpg, Hyp, Hyp(Bzl), Acdt, Ahch, Akch, Actp, Acp, Ach, 3-Apc, 4-Apc, 4-App, Aic, Ana, Ppca, Tha, Cpa, Hle, Aoa, Aha, and Bip,
wherein said one or more peptides inhibits the activity of an HIV virus, whereby said HIV infection is treated.

18. The method in accordance with claim 17, wherein ψ is selected from the group consisting of L-Pro, D-Pro, L-Hyp, D-Hyp, L-Hyp(Bzl), D-Hyp(Bzl), BZA, ADFPA, Acdn, BTD, 4-BZD, Tic, Haic, CPL and PLSP.

19. The method in accordance with claim 17, said one or more peptides or peptide analogs have a formula selected from the group consisting of mpayψwir, mpsaψwir, mpsyψair, mpsyψwar and mpsyψwia, and conservative variants thereof, wherein ψ is selected from the group consisting of L-Pro, D-Pro, L-Hyp, D-Hyp, L-Hyp(Bzl), D-Hyp(Bzl), BZA, ADFPA, Acdn, BTD, 4-BZD, Tic, Haic, CPL and PLSP.

20. The method in accordance with claim 17, said one or more peptides or peptide analogs having the formula mpsyψwir, wherein ψ is selected from the group consisting of L-Pro, D-Pro, L-Hyp, D-Hyp, L-Hyp(Bzl), D-Hyp(Bzl), BZA, ADFPA, Acdn, BTD, 4-BZD, Tic, Haic, CPL and PLSP.

21. The method in accordance with claim 20, wherein ψ is selected from the group consisting of L-Pro, D-Pro, L-Hyp, D-Hyp, L-Hyp(Bzl) and D-Hyp(Bzl).

22. The method in accordance with claim 17, wherein said one or more peptides or peptide analogs inhibit HIV attachment to a cell.

23. The method in accordance with claim 17, wherein said one or more peptides or peptide analogs inhibit HIV entry into a cell.

24. The method in accordance with claim 17, wherein said one or more peptides or peptide analogs bind to different HIV targets.

25. The method in accordance with claim 17, wherein said HIV infection is a drug resistant HIV infection.

26. The method in accordance with claim 17, wherein said HIV infection is a variant HIV infection.

27. The method in accordance with claim 17, further administering one or more of an HIV reverse transcriptase inhibitor or an HIV protease inhibitor.

28. A method of treating HIV infection, said method comprising administering to a subject in need thereof, an effective amount of a peptide or peptide derivative having the formula wqnikdygy and conservative variants thereof, wherein ψ is a turn-promoting amino acid selected from the group consisting of L-Pro, D-Pro, L-Hyp, D-Hyp, L-Hyp(Bzl), D-Hyp(Bzl) and β-turn mimetics.

29. The method in accordance with claim 28, further co-administering an effective amount of one or more peptides or peptide derivatives according to claim 1.

30. A peptide or peptide analog that inhibits the activity of HIV, said peptide or peptide analog identified by a method comprising:
a) contacting in a sample an HIV target protein required for the activity of HIV with a plurality of peptide members of a combinatorial peptide library, wherein each peptide member of said library is attached to a bead, wherein each of said members of said peptide library have a formula selected from the group consisting of
x⁻³x⁻²x⁻¹mpx¹x²ψx⁴x⁵x⁶, x⁻²x⁻¹mpx¹x²ψx⁴x⁵x⁶, x⁻¹mpx¹x²ψx⁴x⁵x⁶, mpx¹x²ψx⁴x⁵x⁶, x⁻¹x¹x²ψx⁴x⁵x⁶x⁷, and conservative variants thereof,
wherein
the lower case letters represent D-forms of the amino acids according to their one-letter code and ψ is a turn-promoting amino acid selected from the group consisting of L-Pro, D-Pro, L-Hyp, D-Hyp, L-Hyp(Bzl), D-Hyp(Bzl) and β-turn mimetics; and
each of x⁻³, x⁻², x⁻¹, x¹, x², x⁴, x⁵, x⁶ and x⁷ are independently selected from L-amino acids, D-amino acids, and L- and D- isomers of Har, Hcy, Hse, Met(O), Met (S-Me), Nle, Tau, Phg, HoPhe, Phe(2-Me), Phe(3-Me), Phe(4-Me), Phe(2-F), Phe(3-F), Phe(4-F), Phe(2-Cl), Phe(3-Cl), Phe(4-Cl), Phe(2-Br), Phe(3-Br), Phe(4-Br), Phe(2-I), Phe(3-I), Phe(4-I), Phe(2-CF₃), Phe(3-CF₃), Phe(4-CF₃), Phe(2-OMe), Phe(3-OMe), Phe(2-NO₂), Phe(3-NO₂), Phe(4-NO₂), Phe(2-CN), Phe(3-CN), Phe(4-CN), Phe(3,4-di OMe), Phe(3,4-di F), Phe(3,5-di F), Phe(2,4-di Cl), Phe(3,4-di Cl), Phe(4-N₃), Phe(4-NH₂), Phe(4-COOH), HoCit, Cit, Orn, 2-Thi, 3-Thi, Chg, Cha, Nal-2, Nal-1, Aib, Acpc, Aad, Asu, 4-Pal, 3-Pal, Pra, Abu, Nva, Dpr, Dbu, Thz, Tyr(Me), Tyr(3,5-di Br), Tyr(3,5-di I), Tyr(3,5-di NO₂), Tyr(3-NO₂), Bug, Bta, Bpa, Dpa, Deg, Dpg, Hyp, Hyp(Bzl), Acdt, Ahch, Akch, Actp, Acp, Ach, 3-Apc, 4-Apc, 4-App, Aic, Ana, Ppca, Tha, Cpa, Hle, Aoa, Aha, and Bip;
b) isolating a bead attached to a peptide member that is bound to said HIV target protein, and
c) sequencing said peptide member.

31. The peptide or peptide analog of claim 30, wherein x1 and x2 are both arginine.

32. The peptide or peptide analog of claim 30, wherein said HIV target protein is an envelope protein.

33. The peptide or peptide analog of claim 30, wherein said peptide binds to an HIV transmembrane subunit gp41.

34. The peptide or peptide analog of claim 30, wherein said HIV target protein is a rapidly mutating protein.

35. The peptide or peptide analog of claim 30, wherein said HIV target protein is from a drug resistant HIV virus.

36. The peptide or peptide analog of claim 30, wherein said identified peptide inhibits HIV activity in vitro with an EC₅₀ of less than 20 μM.

37. A method of treating HIV infection, said method comprising administering to a subject in need thereof, an effective amount of one or more peptides or peptide derivatives of claim 30.

38. A pharmaceutical composition comprising one or more of a peptide or peptide analog of claim 1.

39. The pharmaceutical composition of claim 38, wherein said composition is prepared for topical administration.

40. The pharmaceutical composition of claim 39, wherein said peptide or peptide analog is virucidal.

41. A method for prophylactically or therapeutically inhibiting an HIV infection, the method comprising:
administering to an individual a topical pharmaceutical composition comprising an effective amount of one or more of a peptide or peptide analog of claim 1.

42. The method of claim 41, wherein said pharmaceutical composition is administered to the vagina, the penis the rectum or the mouth of said individual.
